(19) United States
(12) Patent Application Publication
(10) Pub. No.: US 2025/0041237 A1
(43) Pub. Date: Feb. 6, 2025

(54) HEMOGLOBIN-BASED NANOPARTICLES

(71) Applicant: Wisconsin Alumni Research Foundation, Madison, WI (US)

(72) Inventors: Shaoqin Gong, Middleton, WI (US); Jingcheng Zhu, Madison, WI (US)

(21) Appl. No.: 18/790,293

(22) Filed: Jul. 31, 2024

Related U.S. Application Data
(60) Provisional application No. 63/530,237, filed on Aug. 1, 2023.

(51) Int. Cl. A61K 9/51; A61K 31/137; A61K 31/438; A61K 31/505; A61K 31/635; A61K 31/7135; A61K 45/06; A61P 31/04

(52) U.S. Cl. CPC A61K 9/5176; A61K 9/5169; A61K 31/137; A61K 31/438; A61K 31/505; A61K 31/635; A61K 31/7135; A61K 45/06; A61P 31/04

(57) ABSTRACT

A nanoparticle comprising a core and a shell, wherein the core includes an antimicrobial and hemoglobin, the shell includes a polyunsaturated fatty acids (PUFA)-containing cell membrane, and the antimicrobial sensitizes bacteria to oxidant killing.

### CROSS-REFERENCE TO RELATED APPLICATIONS

This application claims the benefit of and priority to U.S. Provisional Appl. No. 63/530,237, filed Aug. 1, 2023, the contents of each of which are incorporated herein by reference in their entirety for any and all purposes.

### FIELD

The present technology relates generally to hemoglobin-based nanoparticles and uses thereof for the treatment of bacterial infections.

### BACKGROUND

Infectious diseases are a growing threat to public health owing to increasing antimicrobial resistance and stagnation in new antibiotic development. Staphylococcus aureus (S. aureus), for example, poses a grave threat to public health. With the emergence of antimicrobial resistance, susceptibility to S. aureus infections has increased dramatically. The gravity of this situation is exemplified by the fact that methicillin-resistant S. aureus (MRSA) is now responsible for more deaths in the United States than AIDS and has been identified by the Centers for Disease Control and Prevention (CDC) as a serious threat. Moreover, it has become increasingly challenging and costly to develop new antibiotics. In response to this dire situation, researchers have explored non-antibiotic antibacterial strategies including immune-modulating approaches. Current immunotherapies against S. aureus infections typically rely on antibodies, which can enhance the opsonization of the bacteria, thereby promoting phagocytosis and clearance of the bacteria. However, these approaches have had limited efficacy in clinical trials, despite promising results in preclinical animal models due to the complex and multifactorial nature of staphylococcal infection pathogenesis.

### SUMMARY

The present technology provides nanoimmunotherapies to sensitize bacterial pathogens to neutrophil killing and simultaneously boost innate immunity. This may be achieved by nanoparticles comprising a core and a shell, wherein the core comprises (or consists essentially of, or consists of) an antimicrobial and hemoglobin, the shell comprises (or consists essentially of, or consists of) a polyunsaturated fatty acids (PUFA)-containing cell membrane, and the antimicrobial sensitizes bacteria to oxidant killing. In any embodiments, the antimicrobial may be a redox enzyme inhibitor, an inhibitor of staphyloxanthin biosynthesis, and/or an antibiotic that sensitize bacteria to oxidant killing. In any embodiments, the PUFA-containing cell membrane may be a red blood cell membrane. Additionally, there are provided pharmaceutical compositions comprising a nanoparticle as described herein, and a pharmaceutically acceptable carrier or excipient.

Thus, in an aspect, a nanoparticle (“NP”) is provided that comprises a core and a shell, wherein the core comprises, consists essentially of, or consists of an antimicrobial and hemoglobin, the shell comprises, consists essentially of, or consists of a polyunsaturated fatty acids (PUFA)-containing cell membrane, and the antimicrobial sensitizes bacteria to oxidant killing.

In any embodiments, the antimicrobial may be a redox enzyme inhibitor or an inhibitor of staphyloxanthin biosynthesis. In any embodiments, the antimicrobial may be naftifine, ALS 4, and/or a bacterial redox enzyme inhibitor. In any embodiments, the antimicrobial may be an inhibitor of thioredoxin reductase.

In any embodiments, the antimicrobial may be auranofin. In any embodiments, the antimicrobial includes about 2.5 wt % to about 60 wt % of the core (based on the total weight of the core). In any embodiments, the nanoparticle may further include molecular oxygen (O₂).

In any embodiments, the PUFA-containing cell membrane may be selected from the group consisting of red blood cell membrane (RBCM), macrophage cell membrane, neutrophil cell membrane, mesenchymal stem cell membrane, and platelet cell membrane.

In any embodiments, the core may comprise, consist essentially of, or consist of naftifine and hemoglobin, wherein the core may be coated with RBCM. In any embodiments, RBCM may include about 30 wt % to about 60 wt % of the nanoparticle (based on the total weight of the nanoparticle).

In any embodiments, the nanoparticle may include about 2.5 wt % to about 40 wt % naftifine based on the total weight of the core. In any embodiments, the hemoglobin includes about 60 wt % to about 97.5 wt % of the core (based on the total weight of the core).

In any embodiments, the core may include a molar ratio of hemoglobin to naftifine of about 1:3 to about 1:300. In any embodiments, the molar ratio of hemoglobin to naftifine may be about 1:10 to about 1:90. In any embodiments, the nanoparticle may have a hydrodynamic diameter of about 20 nm to about 400 nm. In any embodiments, the nanoparticle may have a hydrodynamic diameter of about 40 nm to about 150 nm. In any embodiments, the nanoparticle may have a zeta potential of 0 mV to about −40 mV.

In an aspect, the present technology provides a pharmaceutical composition comprising a nanoparticle as described herein, and a pharmaceutically acceptable carrier or excipient.

In an aspect, the present technology provides methods of treatment comprising administering an effective amount of a nanoparticle of any embodiment herein or an effective amount of a pharmaceutical composition of any embodiment herein to a subject suffering from a bacterial infection, such as antibiotic-resistant bacterial infections.

In any embodiments, the bacterial infection may be caused by bacteria selected from the group consisting of S. aureus, E. coli, P. aeruginosa, C. difficile, E. faecium, and K. pneumonia. In any embodiments, the bacteria may be anti-biotic resistant bacteria. In any embodiments, the bacteria may be S. aureus Newman, methicillin-resistant S. aureus (MRSA), or multidrug-resistant (MDR) S. aureus.

In any embodiments, the effective amount of the nanoparticle may range from about 0.1 mg/kg/day to about 500 mg/kg/day. In any embodiments, the nanoparticle may effect incorporation of PUFA into a membrane of the bacteria, oxidation of hydrogen sulfide produced by the bacteria, and/or potentiate neutrophil-killing of the bacteria causing the bacterial infection. In any embodiments, the bacterial infection may comprise a bacterial biofilm and/or bacterial persisters. In any embodiments, the bacterial infection may include a skin infection, bacteremia, bone infection, gastroenteritis, sinus infection, ear infection, urinary tract infection, endocarditis, and/or pneumonia.

In any embodiments, the method may further comprise administering an effective amount of an antibiotic to the subject before, during, or after administration of the nanoparticle. In any embodiments, the antibiotic may be selected from the group consisting of trimethoprim, rifabutin, and/or sulfamethoxazole.

### BRIEF DESCRIPTION OF THE DRAWINGS

FIGS. 1A-1D are schematic illustrations of illustrative embodiments of the present technology. FIG. 1A is a schematic of an illustrative embodiment of the present nanoparticles and their fabrication. FIG. 1B is a schematic illustration of the various pathways enabled by an illustrative embodiment of the present disclosure, Hb-Naf@RBCM NP, to sensitize S. aureus toward host oxidant killing: (1) The biosynthesis of STX, which S. aureus uses to evade host oxidant killing, is inhibited by naftifine; (2) The Hb-Naf@RBCM NP with an RBCM coating that contains abundant PUFA utilizes S. aureus's unique fatty acid metabolic mechanism to modify the composition of the cell membrane lipid, resulting in an increased susceptibility of the bacteria to host oxidant killing; (3) The presence of ferric Hb assists in decreasing the bacterial H₂S level by catalyzing the oxidation of H₂S, thereby increasing the susceptibility of S. aureus to oxidant killing mediated by neutrophils. FIG. 1C is a schematic illustration of Hb-induced lipid peroxidation in S. aureus with the remodeled lipid composition. FIG. 1D is a schematic illustration of the various mechanisms enabled by the Hb-Naf@RBCM NP to enhance the neutrophilic bactericidal activity against S. aureus: (1) The altered lipid profile of S. aureus cell membrane and formation of ferryl Hb within the infection site can initiate lipid peroxidation in S. aureus, thereby facilitating neutrophil chemotaxis; (2) The Hb within the NPs acts as an oxygen carrier that counters the effects of hypoxia in infected tissues and restores the compromised neutrophil respiratory burst against S. aureus.

FIGS. 2A-2I: FIG. 2A shows the hydrodynamic sizes of Hb, Hb-Naf NP, and Hb-Naf@RBCM NP of the Examples as measured by DLS. FIGS. 2B and 2C show a TEM image of the morphology of Hb-Naf@RBCM NP and TEM images of Hb-Naf NP, respectively. Scale bar: 50 nm and 150 nm. FIGS. 2D and 2E show zeta potentials of various NPs including RBCM-derived vesicles, Hb-Naf NP, and Hb-Naf@RBCM NP as measured by DLS. Data are presented as mean±s.d. (n=3). FIG. 2F shows the effects of different treatments including PBS, Hb, naftifine, Hb-Naf NP, or Hb-Naf@RBCM on STX levels as determined by absorbance at 450 nm in three S. aureus strains (Newman, MRSA, MDRSA). Data are presented as mean±s.d. (n=6). FIG. 2G shows effects of different treatments on MRSA pigmentation as imaged after centrifugation. FIG. 2H shows representative images of the Pb-acetate-soaked paper strips showing brown stains of Pb-acetate quantifying the levels of gaseous H₂S generated from MRSA cultures. Numbers indicate the H₂S production level relative to the PBS group. FIG. 2I shows the H₂S levels in three S. aureus strains treated with PBS, Hb, naftifine, Hb-Naf NP, and Hb-Naf@RBCM were quantified using a WSP5 fluorescent H₂S probe. Data are presented as mean±s.d. (n=6). FIG. 2J shows representative fluorescence images of live MRSA for the different treatment groups including PBS, Hb, naftifine, Hb-Naf NP, or Hb-Naf@RBCM NP. A TICT-based fluorescent H₂S probe was applied to the live MRSA cells. Higher fluorescence intensity indicates a higher level of H₂S. Scale bar: 10 m. FIG. 2K shows the mean fluorescence intensity in FIG. 2J was quantified. The statistical significance was calculated using the PBS group data as the control. Data are presented as mean±s.d. (n=3). Statistical significance was calculated via one-way analysis of variance (ANOVA) with Tukey's post hoc test.

FIGS. 3A-3F show results from a stability study of the Hb-Naf NP and Hb-Naf@RBCM NP. The NPs and their sizes and polydispersity index (PDI) were monitored by dynamic light scattering (DLS) at different time points for an aqueous solution at 4° C. (FIGS. 3A and 3B), conditions mimicking the microenvironment of the infected tissues (i.e., pH 6.5, 150 μM H₂O₂, 37° C.) (FIGS. 3C and 3D), and in serum-containing media (i.e., PBS+10% fetal bovine serum, 37° C.) (FIGS. 3E and 3F). Data are presented as mean±s.d. (n=3). Statistical significance of the data collected at 48 h was calculated via a two-tailed Student's t-test. Time-dependent stability of the Hb-Naf NP and Hb-Naf@RBCM NP in (a, b) an aqueous solution at 4° C.; (c, d) with their sizes and polydispersity indexes (PDI) characterized by dynamic light scattering (DLS).

FIGS. 4A and 4B show the release profiles of naftifine and hemoglobin (Hb), respectively, from Hb-Naf@RBCM NP with or without (w/o) the presence of three strains of S. aureus. Data are presented as mean±s.d. (n=3 biologically independent samples per group).

FIGS. 5A-5L: FIG. 5A is a schematic illustration of the antimicrobial assay on planktonic bacteria. The viability of S. aureus Newman (FIG. 5B), MRSA (FIG. 5C), and MDRSA (FIG. 5D) under different treatments was determined respectively by plating the bacteria on the MHB agar plates. Data are presented as mean±s.d. (n=6). FIG. 5E is a schematic illustration of the antimicrobial assay on S. aureus persisters. The viability of S. aureus Newman persisters (FIG. 5F), MRSA persisters (FIG. 5G), and MDRSA persisters (FIG. 5H) with different treatments was determined respectively by plating the bacteria on the MHB agar plates. Data are presented as mean±s.d. (n=6). FIG. 5I is a schematic illustration of the antimicrobial assay on biofilms. The viability of S. aureus Newman (FIG. 5J), MRSA (FIG. 5K), and MDRSA (FIG. 5L) in biofilm with different treatments was determined respectively by dislodging the biofilm-encased bacteria and plating the bacteria on the MHB agar plates. Data are presented as mean±s.d. (n=6). Statistical significance was calculated via one-way analysis of variance (ANOVA) with Tukey's post hoc test.

FIGS. 6A-6K: FIGS. 6A-6C show confirmation of the persistence of stationary-phase S. aureus cells assessed by studying agar plated stationary y-phase cells of S. aureus Newman, MRSA, and MDRSA treated with 100×MIC ciprofloxacin or 100×MIC vancomycin for 4 h. Data are presented as mean±s.d. (n=3). FIG. 6D shows the effect of Hb-Naf@RBCM NP on the H₂S level of S. aureus persisters. Data are presented as mean±s.d. (n=6). Statistical significance was calculated via one-way ANOVA with Tukey's post hoc test. FIG. 6E shows biofilm characterization of biofilms of different strains of S. aureus, where bacterial numbers were counted by colony-forming unit (CFU) assays of three strains of S. aureus in biofilms, and they were characterized by crystal violet assay. FIGS. 6F-6H show the viability of S. aureus Newman, MRSA, and MDRSA in biofilm with different treatments was determined respectively by plating the bacteria on the MHB agar plates. FIGS. 6I-6K show the effect of Hb-Naf@RBCM NP on the antimicrobial ability with or without human neutrophils (hNEs) determined in MRSA plankton, persisters, and biofilms by plating the bacteria on the MH agar plates, and the anti-biofilm capability was determined by crystal violet assay. Data are presented as mean±s.d. (n=6). Statistical significance was calculated via one-way (ANOVA) with Tukey's post hoc test.

FIGS. 7A-7F: FIG. 7A shows representative SEM images showing the morphology of MRSA treated with PBS, Hb, naftifine, Hb-Naf NP, or Hb-Naf@RBCM NP respectively under a condition (pH 6.5 and 150 μM H₂O₂) mimicking the infectious microenvironment. FIG. 7B is representative confocal laser scanning microscopy (CLSM) images of MRSA with different treatments for 2 h in the presence of ROS-sensitive dye carboxy-H₂DCFDA, reflects the ROS level in MRSA (green fluorescence). Scale bar: 25 μm. FIG. 7C shows quantification of mean fluorescence intensity per bacterium in FIG. 7B. The effect of the Hb-Naf@RBCM NP on the lipid peroxide (LPO) level of MRSA studied using C11-BODIPY, a lipid peroxidation-sensitive dye, is shown in terms of quantification of mean fluorescence intensity per bacterium (FIG. 7D) and the lipid peroxides level in MRSA which are representative CLSM images of MRSA with different treatments for 2 h in the presence of (green fluorescence). Scale bar: 100 μm (FIG. 7E) as well as the images taken bright field and a green fluorescence channel and 5 μm (FIG. 7F).

FIG. 8A-8C show the effect of the Hb-Naf@RBCM NP on the MDA-modified proteins level of S. aureus. The levels of MDA-modified proteins were measured by ELISA, in MRSA (FIG. 8A), S. aureus Newman (FIG. 8B), and MDRSA (FIG. 8C) treated with PBS/pH 6.5+H₂O₂, Hb/pH 6.5+H₂O₂, naftifine/pH 6.5+H₂O₂, Hb-Naf NPs/pH 6.5+H₂O₂, Hb-Naf@RBCM NP/pH 6.5+H₂O₂, Hb-Naf@RBCM NP/H₂O₂, Hb-Naf@RBCM NP/pH 6.5, or Hb-Naf@RBCM NP for 2 h, respectively. Data are presented as mean±s.d. (n=6). Statistical significance was calculated via one-way analysis of variance (ANOVA) with Tukey's post hoc test.

FIG. 9A is a schematic illustration of the transwell cell migration assay. Cells were seeded in the upper chamber while the cell membrane of S. aureus with different treatments was added in the lower chamber. A transwell cell migration assay was used to determine the chemotaxis of murine neutrophils or human neutrophils to the cell membrane of S. aureus Newman (murine neutrophils) (FIG. 9B), MRSA (murine neutrophils) (FIG. 9C), MDRSA (murine neutrophils) (FIG. 9D) aid MRSA (human neutrophils) (FIG. 9E) with various treatments. Data are presented as mean±s.d. (n=6). Statistical significance was calculated via one-way analysis of variance (ANOVA) with Tukey's post hoc test.

FIGS. 10A-10I: FIG. 10A shows measurements of the oxygen release profiles of Hb-Naf@RBCM NP and controls. Measurement of oxygen concentration in PBS was made after adding oxygen-pre-saturated Hb, Hb-Naf@RBCM, and BSA-Naf@RBCM with an equivalent amount of 8 mg mL⁻¹ Hb. Data are presented as mean±s.d. (n=3). Statistical significance was calculated via one-way analysis of variance (ANOVA) with Tukey's post hoc test. FIG. 10B shows representative CLSM images of NIH 3T3 cells with different treatments for 1 h in the presence of a red hypoxia detection probe (ROS-ID® Hypoxia/Oxidative stress detection kit). The red fluorescence reflects high hypoxia levels in cells. Cell nuclei were stained by Hoechst 33342 (blue). Scale bar: 100 m. FIG. 10C shows the mean fluorescence intensity of hypoxia with different treatments under hypoxic conditions. The viability of S. aureus Newman (FIG. 10D), MRSA (FIGS. 10E, 10G), and MDRSA (FIG. 10F) with different treatments under in-vitro hypoxic conditions was determined by plating the bacteria on MHB agar plates. Data are presented as mean±s.d. (n=6). FIG. 10H shows that extracellular superoxide generation by neutrophils stimulated with PMA (200 nM) was assessed by a superoxide anion assay kit. Data are presented as mean±s.d. (n=6). FIG. 10I shows that the Intracellular ROS level of neutrophils stimulated with zymosan A was determined by CM-H₂DCFDA staining and flow cytometry. Data are presented as mean±s.d. (n=6). Statistical significance was calculated via one-way analysis of variance (ANOVA) with Tukey's post hoc test.

FIG. 11 shows resistance development of MRSA after continuous treatment of neutrophils+Hb-Naf@RBCM NP. Data are presented as mean±s.d. (n=3).

FIGS. 12A-12E: FIG. 12A is a schematic illustration of the experimental procedures for the biodistribution study in the MRSA thigh infection model. FIG. 12B shows that ex vivo fluorescence images represent the in vivo biodistribution of Cy5.5-labelled Hb-Naf NP and Hb-Naf@RBCM NP in mice with MRSA thigh infection 6 h and 24 h after NP administration. FIGS. 12C and 12D show the biodistribution of the Hb-Naf NP and Hb-Naf@RBCM NP in the infected thigh in mice at 6 h and 24 h, respectively. Data are presented as mean±s.d. (n=3). The statistical significance was calculated via a two-tailed Student's t-test. FIG. 12E shows that bacteria burden of mice with MRSA thigh infection.

FIGS. 13A-13C shows representative immunostaining images and plots showing neutrophil chemotaxis following Hb-Naf@RBCM NP treatment in infected thighs.

FIGS. 14A-14C: FIG. 14A is a schematic illustration of the experimental procedures for the antimicrobial efficacy study in the MRSA lung infection model. FIG. 14B shows representative histological images for lung after bilateral intranasal inoculation of MRSA at different time points. FIG. 14C shows that bacteria burden of mice with MRSA lung infection was determined by through serial dilution and plate counting of homogenized organs. Data are presented as mean±s.d. (n=6). Statistical significance was calculated via one-way analysis of variance (ANOVA) with Tukey's post hoc test.

FIGS. 15A-15D: FIG. 15A shows experimental procedures for the antimicrobial efficacy study in the S. aureus MRSA peritonitis model. Survival (FIG. 15B) and body weight (FIG. 15C) analysis of the mice in the MRSA peritonitis model. Data are presented as mean±s.d. (n=8). Statistical significance was calculated via a log-rank test. FIG. 15C shows therapeutic efficacy of the Hb-Naf@RBCM NP in the mouse MRSA peritonitis model. The CFU in the heart, liver, spleen, lung, kidneys, and ascites was determined 12 h after the infection through serial dilution and plate counting of homogenized organs. Data are presented as mean±s.d. (n=6). Statistical significance was calculated via one-way analysis of variance (ANOVA) with Tukey's post hoc test.

FIGS. 16A-16D: FIG. 16A shows experimental procedures for the antimicrobial efficacy study in the MDRSA bacteremia model. Survival (FIG. 16B) and body weight (FIG. 16C) analysis of the mice in the MDRSA bacteremia model with various treatments. Data are presented as mean±s.d. (n=8). Statistical significance was calculated via a log-rank test. FIG. 15D show therapeutic efficacy of the Hb-Naf@RBCM NP in the mouse MDRSA bacteremia model. The CFU in the heart, liver, spleen, lung, kidneys, and blood was determined 12 h after the infection through serial dilution and plate counting of homogenized organs or blood. Data are presented as mean±s.d. (n=6). Statistical significance was calculated via one-way analysis of variance (ANOVA) with Tukey's post hoc test.

FIG. 17 shows representative histological images for tissue sections stained with hematoxylin and eosin from the healthy mice (control) and the infected mice treated with PBS or Hb-Naf@RBCM NP.

FIGS. 18A-18C show in vitro cytotoxicity assessment of the Hb-Naf@RBCM NP for RAW 264.7 cells, HEK 293 cells, and NIH 3T3 cells via the MTT assay. Data are presented as mean±s.d. (n=3). Statistical significance was calculated via one-way ANOVA with Tukey's post hoc test. Data are compared to the untreated group.

FIG. 19 shows the blood biochemical parameter panel for healthy mice subjected to triple injections of PBS, Hb-Naf NP, and Hb-Naf@RBCM NP, respectively. Blood biochemistry analysis of healthy mice treated with PBS, Hb-Naf NP (125 mg kg⁻¹ Hb, 25 mg kg⁻¹ naftifine), or Hb-Naf@RBCM NP (125 mg kg⁻¹ Hb, 25 mg kg⁻¹ naftifine) on Day 0, Day 2 and Day 4. A total of three injections were applied to each mouse. Fresh whole blood of these mice was collected on Day 7. BUN, blood urea nitrogen. CRE, creatinine. ALT, alanine aminotransferase. ALP, alkaline phosphatase. AST, aspartate aminotransferase. TBIL, total bilirubin. GLU, glucose. CA, total calcium. TP, total protein. ALB, albumin. GLOB, globulin. Data are presented as mean±s.d. (n=3). Statistical significance was calculated via one-way ANOVA with Tukey's post hoc test.

FIG. 20 shows H&E staining for major organs from healthy mice subjected to triple injections of PBS, Hb-Naf NP, and Hb-Naf@RBCM NP, respectively. Representative histological images for mice (n=3) treated with i.v. injection of PBS, Hb-Naf NP (125 mg kg⁻¹ Hb, 25 mg kg⁻¹ naftifine), or Hb-Naf@RBCM NP (125 mg kg⁻¹ Hb, 25 mg kg⁻¹ naftifine) on Day 0, Day 2 and Day 4. A total of three injections were applied to each mouse. On Day 7, organs were collected, sectioned, stained, and imaged. Scale bar: 100 m.

FIGS. 21A-21C show the proinflammatory levels of PBS-injected and Hb-Naf@RBCM NP-injected healthy mice. The levels of serum proinflammatory cytokines (FIG. 21A) IL-10, FIG. 21B) TNF-α, and (FIG. 21C) IL-6, tested by ELISA in healthy mice treated with PBS or Hb-Naf@RBCM NP (250 mg kg⁻¹ Hb and 50 mg kg⁻¹ naftifine). Data are presented as mean±s.d. (n=3). Statistical significance was calculated via a two-tailed Student's t-test.

FIGS. 22A-22B: FIG. 22A shows representative CLSM images of macrophages incubated with Cy5-labeled Hb-Naf@RBCM NP. The nuclei were stained with Hoechst 33342 (blue) and Hb-Naf@RBCM NPs were labeled with Cy5 (red). Scale bar: 5 m. FIG. 22B shows the mean fluorescence intensity of Cy5 signal per cell was quantified by NIS-Element. Data are presented as mean±s.d. (n=3). Statistical significance was calculated via one-way ANOVA with Tukey's post hoc test.

### DETAILED DESCRIPTION

### Definitions

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term—for example, “about 10 wt. %” would be understood to mean “9 wt. % to 11 wt. %.” It is to be understood that when “about” precedes a term, the term is to be construed as disclosing “about” the term as well as the term without modification by “about”—for example, “about 10 wt. %” discloses “9 wt. % to 11 wt. %” as well as disclosing “10 wt. %.”

The phrase “and/or” as used in the present disclosure will be understood to mean any one of the recited members individually or a combination of any two or more thereof—for example, “A, B, and/or C” would mean “A, B, C, A and B, A and C, B and C, or the combination of A, B, and C.”

“Antimicrobial” as used herein refers to compounds that sensitize bacteria to oxidant killing in NP of the present technology. Additionally, the present antimicrobials exhibit good protein binding affinity, e.g., >30%. Thus, antimicrobials of the present technology include compounds that, e.g., are staphyloxanthin inhibitors or bacterial redox enzyme inhibitors, and traditional antibiotics that, regardless of their mechanism of action, also sensitize bacteria to oxidant killing. For example, antibiotics such as β-lactams, quinolones and aminoglycosides lead to higher levels of reactive oxygen species within the targeted bacteria and are therefore antimicrobials of the present technology.

“Bacterial redox enzyme inhibitor” as used herein refers to inhibitors of enzymes involved in reduction/oxidation pathways that are protective of a bacterium from oxidative damage, e.g., thioredoxin reductase which catalyzes the reduction of thioredoxin, a protein that, e.g., contributes to keeping disulfide bonds reduced in bacteria.

As used herein, the terms “effective amount” or “therapeutically effective amount,” or “pharmaceutically effective amount” refer to a quantity sufficient to achieve a desired therapeutic and/or prophylactic effect, e.g., an amount which results in the full or partial amelioration (e.g., inhibiting, relieving, climinating, or slowing progression of one or more symptoms) of disease or disorders or symptoms associated therewith in a subject (e.g., a mammal) in need thereof. In the context of therapeutic or prophylactic applications, the amount of a composition administered to the subject will depend on the type and severity of the disease and on the characteristics of the individual, such as general health, age, sex, body weight, and tolerance to drugs. It will also depend on the degree, severity and type of disease. A person of ordinary skill in the art will be able to determine appropriate dosages depending on these and other factors. The compositions can also be administered in combination with one or more additional compounds. Multiple doses may be administered. Additionally or alternatively, multiple therapeutic compositions or compounds may administered. In the methods described herein, the compounds may be administered to a subject having one or more signs or symptoms of a disease or disorder described herein.

“Polyunsaturated fatty acid” (or “PUFA”) as used herein refers to C₁₄ to C₂₄ fatty acids having at least two carbon-carbon cis double bonds. In any embodiments, the PUFA may be a C₁₄, C₁₆, C₁₈, C₂₀, C₂₂, or C₂₄ PUFA or is a PUFA having a range between and/or including any two of the foregoing numbers of carbon, e.g., C₁₈ to C₂₂ PUFA. In any embodiments, PUFA may include an ω3- and/or ω6-PUFA. PUFA may include, e.g., ω3-PUFA such as eicosapentaenoic acid (EPA, 20:5(n-3)) and/or docosahexanoic acid (DHA, 22:6(n-3)), ω6-PUFA such as α-linoleic acid (ALA, 18:2(n-3)).

“Polyunsaturated fatty acids-containing cell membrane” (or “PUFA-containing cell membrane”) as used herein refers to cell membranes having about 5 wt % to about 55 wt % PUFA based on the total cell membrane fatty acid content. In any embodiments, the PUFA content may be about 5 wt %, about 10 wt %, about 15 wt %, about 20 wt %, about 25 wt %, about 30 wt %, about 35 wt %, about 40 wt %, about 45 wt %, about 50 wt %, about 55 wt %, or a range between and/or including any two of the foregoing values, e.g., about 25 wt % to about 50 wt % PUFA.

The terms “preventing” and “prophylaxis” as used herein refer to administering a pharmaceutical compound or medicament or a composition including the pharmaceutical compound or medicament to a subject before a disease, disorder, or condition fully manifests itself, to forestall the appearance and/or reduce the severity of one or more symptoms of the disease, disorder, or condition. The person of ordinary skill in the art recognizes that the term “prevent” is not an absolute term. In the medical art it is understood to refer to the prophylactic administration of a drug to diminish the likelihood or seriousness of a disease, disorder or condition, or a symptom thereof, and this is the sense that such terms are used in this disclosure.

As used herein, the term “subject” refers to any animal that can experience sepsis, such as a mammal or a bird. In any embodiments, the mammal may be selected from primates, dogs, cats, rodents, horses, cattle, or pigs. In any embodiments, the subject (i.e., primate subject) may be a human.

“Treating,” “treat,” “treated,” or “treatment” as used herein covers the treatment of a disease or disorder described herein (e.g., AD), in a subject, such as a human, and includes: (i) inhibiting or preventing a disease or disorder, i.e., arresting its development; (ii) relieving a disease or disorder, i.e., causing regression of the disorder; (iii) slowing progression of the disorder; and/or (iv) inhibiting, preventing, relieving, ameliorating, or slowing progression of one or more symptoms of the disease or disorder. Symptoms may be assessed by methods known in the art or described herein, for example, biopsy, histology, and blood tests to determine relevant enzyme levels, metabolites or circulating antigen or antibody (or other biomarkers), quality of life questionnaires, patient-reported symptom scores, and imaging tests.

“Ameliorate,” “ameliorating,” and the like, as used herein, refer to inhibiting, relieving, eliminating, or slowing progression of one or more symptoms.

### Nanoparticles

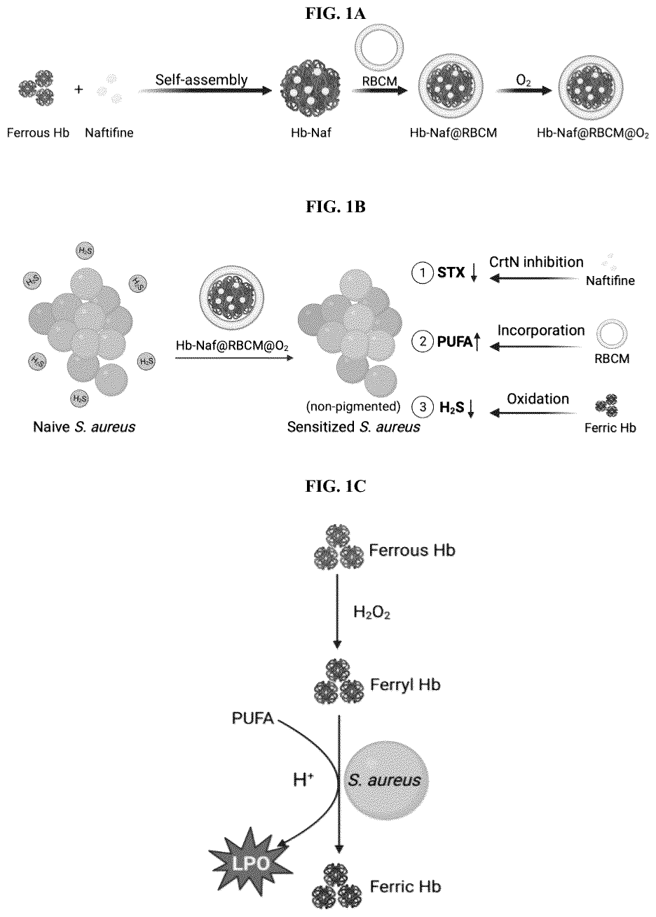

The present technology provides nanoimmunotherapies for use in the treatment of bacterial infections, including those involving antibiotic-resistant bacteria, persisters, and biofilms, among others. Provided herein are nanoparticles (NPs) that include a core and a shell. The core includes an antimicrobial and hemoglobin, wherein the antimicrobial sensitizes bacteria to oxidant killing. The shell includes a polyunsaturated fatty acid (PUFA)-containing cell membrane. While not wishing to be bound by theory, it is believed that when exposed to the acidic and H₂O₂-rich infectious microenvironment, hemoglobin in the NPs is activated to generate ferric hemoglobin and highly toxic ferryl hemoglobin (FIG. 1C). Ferric hemoglobin helps lower the bacterial H₂S level by catalyzing H₂S oxidation, thus sensitizing bacteria (e.g., S. aureus) to neutrophil-mediated oxidant killing (FIG. 1B). In tandem with the S. aureus sensitizing effects enabled by the antimicrobial (e.g., naftifine), RBCM, and ferric hemoglobin (FIG. 1B), ferryl hemoglobin can catalyze lipid peroxidation (FIG. 1C), a process where oxidants such as free radicals attack PUFA present in the bacterial cell membrane incorporated from the PUFA-containing cell membrane of the present NPs. The accumulation of lipid peroxides (LPO) enhances neutrophil chemotaxis, especially long-range immune detection. Moreover, hemoglobin in the NPs serves as a natural oxygen carrier that mitigates the effects of hypoxia in infected tissues and restores the antimicrobial function of neutrophils by enhancing the impaired neutrophil respiratory burst against bacteria such as S. aureus. Thus, the bactericidal activity of neutrophils can be significantly enhanced (FIG. 1D). This dual strategy of promoting the antimicrobial ability of neutrophils via enhanced chemotaxis and improved respiratory burst, while sensitizing bacteria (such as S. aureus) to host oxidant-killing, proved to be highly effective as shown herein.

In any embodiments of the present NPs, the antimicrobial may be a bacterial redox enzyme inhibitor or an inhibitor of staphyloxanthin biosynthesis, or even an antibiotic, e.g., one that also leads to increased sensitivity to oxidant-killing. For example, the antimicrobial may be an inhibitor of staphyloxanthin biosynthesis such as naftifine and/or ALS 4. In some embodiments, the antimicrobial is naftifine. Alternatively, the antimicrobial may be a bacterial redox enzyme inhibitor, e.g., an inhibitor of thioredoxin reductase. Thus, in any embodiments, the antimicrobial may be auranofin. In any embodiments, the present NP may include an antibiotic and hemoglobin, where in some such embodiments the NP may provide enhanced permeation and/or retention of the antibiotic at the infection site.

In any embodiments of the present technology, the antimicrobial may make up about 2.5 wt % to about 60 wt % of the core (based on the total weight of the core). For example, the antimicrobial may be about 2.5 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, or a range between and/or including any two of the foregoing values. Thus, in some embodiments, the antimicrobial may comprise about 5 wt % to about 35 wt % of the core. In some embodiments, the antimicrobial is naftifine and may make up, e.g., about 2.5 wt % to about 40 wt % of the core.

In any embodiments of the present nanoparticles, the hemoglobin may be human or non-human hemoglobin, e.g., avian or (non-human) mammalian hemoglobin such as bovine or pig hemoglobin. In any embodiments, the hemoglobin may make up about 40 wt % to about 97.5 wt % of the core (based on the total weight of the core), e.g., about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, or about 97.5 wt %, or a range between and/or including any two of the foregoing values. Thus, in any embodiments, the hemoglobin may make up about 70 wt % to about 90 wt % of the core. In any embodiments, the NPs may further include O₂, e.g., bound to the hemoglobin.

In any embodiments of the present nanoparticles, the core may include a molar ratio of hemoglobin to antimicrobial of about 1:3 to about 1:300. In any embodiments, the antimicrobial may be as described herein, I, naftifine, ALS 4, auranofin, or the like. Thus, the core may include a molar ratio of hemoglobin to antimicrobial of about 1:3, about 1:4, about 1:5, about 1:10, about 1:15, about 1:20, about 1:25, about 1:30, about 1:35, about 1:40, about 1:45, about 1:50, about 1:55, about 1:60, about 1:65, about 1:70, about 1:80, about 1:90, about 1:100, about 1:125, about 1:150, about 1:175, about 1:200, about 1:225, about 1:250, about 1:275, about 1:300, or a range between and/or including any of the forgoing values. For example, the core may include naftifine at any of the foregoing molar ratios or ranges, such as about 1:10 to about 1:90 or about 1:20 to about 1:40. In any embodiments, the molar ratio of antimicrobial (e.g., naftifine) to hemoglobin may be about 1:30.

In the present NPs, the PUFA-containing cell membrane may be selected from the group consisting of red blood cell membrane (RBCM), macrophage cell membrane, neutrophil cell membrane, mesenchymal stem cell membrane, and platelet cell membrane. In any embodiments, the PUFA-containing cell membrane may be RBCM. The PUFA-containing cell membrane may be about 30 wt % to about 60 wt % of the nanoparticle based on the total weight of the nanoparticle, e.g., about 30 wt %, about 35 wt %, about 40 wt %, about 45 wt %, about 50 wt %, about 55 wt %, about 60 wt %, or a range between and/or including any two of the foregoing values. Hence the PUFA-containing cell membrane may be, e.g., about 40 wt % to about 50 wt % RBCM.

The nanoparticles of the present technology may be prepared in a range of sizes, for example, having a hydrodynamic diameter of about 20 nm to about 400 nm, including about 20 nm, about 30 nm, about 40 nm, about 50 nm, about 60 nm, about 70 nm, about 80 nm, about 90 nm, about 100 nm, about 110, nm, about 120 nm, about 130 nm, about 140 nm, about 150 nm, about 175 nm, about 200 nm, about 225 nm, about 250 nm, about 275 nm, about 300 nm, about 350 nm, about 400 nm, or a range between and/or including any two of the foregoing values. In any embodiments, the NPS may have a hydrodynamic diameter of about 40 nm to about 150 nm.

The zeta potential of the present nanoparticles varies. The NPs may have a zeta potential of 0 mV to about −40 mV, e.g., about 0 mV, about −5 mV, about −10 mV, about −20 mV, about −25 mV, about −30 mV, about −35 mV, about −40 mV, or a range between and/or including any two of the foregoing values. For example, in any embodiments, the zeta potential of the present NPs may be about −15 mV to about −35 mV or about −20 mV to about −30 mV.

### Pharmaceutical Compositions

In another aspect, the present technology provides pharmaceutical compositions including any of the NPs described herein and a pharmaceutically acceptable carrier or excipient.

The compositions described herein can be formulated for various routes of administration, for example, by parenteral, intravitreal, intrathecal, intracerebroventricular, rectal, nasal, vaginal administration, direct injection into the target organ, or via implanted reservoir. Parenteral or systemic administration includes, but is not limited to, subcutaneous, intravenous, intraperitoneal, and intramuscular injections. The following dosage forms are given by way of example and should not be construed as limiting the instant present technology.

Injectable dosage forms generally include solutions or aqueous suspensions which may be prepared using a suitable dispersant or wetting agent and a suspending agent so long as such agents do not interfere with formation of the nanoparticles described herein. Injectable forms may be prepared with acceptable solvents or vehicles including, but not limited to sterilized water, phosphate buffer solution, Ringer's solution, 5% dextrose, or an isotonic aqueous saline solution.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference. Exemplary carriers and excipients may include but are not limited to USP sterile water, saline, buffers (e.g., phosphate, bicarbonate, etc.), and/or tonicity agents (e.g., glycerol).

### Methods and Uses

In an aspect, the present technology provides methods of treatment that include administering an effective amount of any of the nanoparticles described herein or an effective amount of a pharmaceutical composition thereof to a subject suffering from a bacterial infection. In any embodiments of the present methods, the bacterial infection may be caused by bacteria such as S. aureus, E. coli, P. aeruginosa, C. difficile, E. faecium, K. pneumonia, or a combination of any two or more thereof. The bacteria may also be anti-biotic resistant bacteria. In any embodiments, the bacteria may be S. aureus Newman, methicillin-resistant S. aureus (MRSA), and/or multidrug-resistant (MDR) S. aureus. In any embodiments of the method, the bacterial infection may be a skin infection, bacteremia, bone infection, gastroenteritis, sinus infection, ear infection, urinary tract infection, endocarditis, and/or pneumonia. In any embodiments, the bacterial infection may include a bacterial biofilm and/or bacterial persisters.

In any embodiments of present methods, the effective amount of the nanoparticle ranges from about 0.1 mg/kg/day to about 500 mg/kg/day, or any amount or range therein. Specific dosages may be adjusted depending on conditions of disease, the age, body weight, general health conditions, sex, and diet of the subject, dose intervals, administration routes, excretion rate, and combinations of drug conjugates. Any of the above dosage forms containing effective amounts are well within the bounds of routine experimentation and therefore, well within the scope of the instant present technology. By way of example only, such dosages may be used to administer effective amounts of the present nanoparticles to the patient and may include an amount in mg/kg/day of about 0.1, about 0.5, about 1.0, about 2.0, about 3.0, about 4.0, about 5.0, about 10, about 15, about 20, about 25, about 30, about 40, about 50, about 60, about 70, about 80, about 90, about 100, about 125, about 150, about 175, about 200, about 225, about 250, about 275, about 300, about 325, about 350, about 375, about 400, about 450, or a range between and/or including any two of the forgoing values (such as about 0.1 mg/kg/day to about 100 mg/kg/day). Such amounts may be administered parenterally as described herein and may take place over a period of time including but not limited to about 5 minutes, about 10 minutes, about 20 minutes, about 30 minutes, about 45 minutes, about 1 hour, about 2 h, about 3 h, about 5 h, about 10 h, about 12 h, about 15 h, about 20 h, about 24 h, or a range between and/or including any of the foregoing values. The frequency of administration may vary, for example, once per day, per 2 days, per 3 days, per week, per 10 days, per 2 weeks, or a range between and/or including any of the foregoing frequencies. More frequent administration is also possible. Alternatively, the compositions may be administered once per day on 2, 3, 4, 5, 6, or 7 consecutive days. A complete regimen may thus be completed in only a few days or over the course of 1, 2, 3, 4, or more weeks.

In any embodiments of the present methods, the nanoparticle may effect incorporation of PUFA into a membrane of the bacteria, oxidation of hydrogen sulfide produced by the bacteria, and/or potentiate neutrophil-killing of the bacteria causing the bacterial infection.

The present methods may further include administering an effective amount of an antibiotic to the subject before, during, or after administration of the nanoparticle(s). For example, the antibiotic may be trimethoprim, rifabutin, and/or sulfamethoxazole.

The examples herein are provided to illustrate advantages of the present technology and to further assist a person of ordinary skill in the art with preparing or using the nanoparticles compositions of the present technology. To the extent that the compositions include ionizable components, salts such as pharmaceutically acceptable salts of such components may also be used. The examples herein are also presented in order to more fully illustrate the preferred aspects of the present technology. The examples should in no way be construed as limiting the scope of the present technology, as defined by the appended claims. The examples can include or incorporate any of the variations or aspects of the present technology described above. The variations or aspects described above may also further each include or incorporate the variations of any or all other variations or aspects of the present technology.

### EXAMPLES

### General: Materials and Methods

Materials. Hemoglobin (Hb) from bovine blood, bovine serum albumin (BSA), naftifine hydrochloride, lead(II) acetate trihydrate, WSP5, phorbol 12-myristate 13-acetate (PMA), and zymosan A were purchased from Sigma-Aldrich, USA. Other reagents including buffers and culture media were purchased from Thermo Fisher Scientific and used as received.

Cell and bacteria culture. Mouse neutrophils were isolated by Percoll-based density gradient centrifugation and used freshly. Human neutrophils were purchased from iQ Bioscience, USA. Murine macrophage cell line RAW 264.7 (ATCC TIB-71) was maintained in RPMI-1640 supplemented with 10% FBS (v/v), 100 U/mL penicillin, and streptomycin at 37° C. in a 5% CO₂ incubator. Mouse embryonic fibroblast cell line NIH 3T3 (ATCC CRL-1658) and human embryonic kidney cell line HEK 293 (ATCC CRL-3216) were cultured in DMEM supplemented with 10% FBS (v/v), 100 U/mL penicillin and streptomycin at 37° C. in a 5% CO₂ incubator. S. aureus Newman, MRSA (ATCC 33591), and MDRSA (ATCC BAA-44) were grown in Mueller Hinton Broth (MHB) at 37° C. with aeration. According to the data from ATCC, MDRSA is resistant to the following antibiotics: azithromycin, cefoxitin, ciprofloxacin, clindamycin, doxycycline, erythromycin, gentamicin, penicillin, rifampin, tetracycline.

Preparation of RBC membrane (RBCM)-derived vesicles. RBCM-derived vesicles were prepared via hypotonic hemolysis. Erythrocytes from ICR mouse blood were centrifuged at 4° C., washed three times with 1×PBS, and then subjected to hypotonic treatment in 0.25×PBS at 4° C. for 1 hour. After 5 times of centrifugation at 16,099 g for 10 minutes to remove hemoglobin, the pellet was resuspended in deionized (DI) water for sonication. Subsequently, the vesicles were extruded through polycarbonate membrane (400 nm) using a mini extruder. Finally, the concentration of membrane protein was quantified by bicinchoninic acid (BCA) assay.

Fabrication of the Hb-Naf nanoparticle (NP). The Hb-Naf NP was prepared by adding a naftifine ethanol solution (20 mg/mL) to a Hb (2 mg/mL) aqueous solution with varying volume ratios under constant stirring. The resulting Hb-Naf NP was then purified by centrifugation (16,099 g, 5 min). The Hb: naftifine molar ratio of 1:30 resulted in the highest drug loading efficiency for naftifine and was selected for subsequent studies.

Fabrication of the Hb-Naf@RBCM NP. To prepare the RBCM-coated Hb-Naf NP (i.e., Hb-Naf@RBCM), the Hb-Naf NP was coated with RBCM-derived vesicles in a 1:1 membrane protein to nanoparticle weight ratio. The mixture was sequentially extruded through polycarbonate porous membranes with pore sizes of 200 nm and then 100 nm using a mini extruder.

Characterization of the NPs. The hydrodynamic size and zeta potential of the NPs were measured using dynamic light scattering (DLS) with a ZetaSizer Nano ZS90 (Malvern Instruments, USA). The morphology of the Hb-Naf@RBCM NP was tested by transmission electron microscopy (TEM) using an FEI Tecnai G2 F30 TWIN 300 KV microscope. The stability of the Hb-Naf NP and Hb-Naf@RBCM NP was evaluated by monitoring their particle size using DLS over 48 h. The loading content and efficiency of naftifine and Hb were quantified by reversed-phase high-performance liquid chromatography (HPLC, Elite LaChrom, Hitachi) equipped with UV detection at 254 nm for naftifine and 405 nm for Hb.

Quantification of the staphyloxanthin (STX) levels in S. aureus. S. aureus Newman, MRSA, or MDRSA were treated with PBS, Hb, naftifine, Hb-Naf NP, or Hb-Naf@RBCM NP with an equivalent Hb concentration of 200 g mL-1 and naftifine concentration of 40 g mL-1 in Mueller Hinton Broth (MHB) for 24 h at 37° C. under shaking at 250 rpm. After the incubation period, the bacterial cultures were centrifuged and washed twice with 10 mM PBS. The optical density (OD) values obtained from different cultures were normalized. The pigment was extracted three times using methanol, and the resulting solution was then brought up to a total volume of 1 mL. The STX level was determined by measuring the absorbance of the solution at 450 nm using a NanoDrop.

H₂S level measurement. H2S measurement was conducted using paper strips saturated with 2% lead acetate, positioned above the S. aureus culture in a cultural tube. Overnight bacteria cultures (S. aureus Newman, MRSA, or MDRSA) were diluted 1:50 in MHB. The diluted bacteria cultures were treated with PBS, Hb, naftifine, Hb-Naf NP, or Hb-Naf@RBCM NP (200 g mL-1 Hb, 40 g mL-1 naftifine) for 12 h. Subsequently, the stained paper strips were scanned using Gel Doc XR+ System (Bio-Rad Laboratories, USA). Fluorescent probes were also used to detect the H2S levels. A twisted internal charge transfer (TICT)-based fluorescent H2S probe (Enamine Ltd, USA) was added to the bacterial inoculums pretreated with Hb, naftifine, Hb-Naf NP, or Hb-Naf@RBCM NP (200 g mL⁻¹ Hb, 40 g mL⁻¹ Naftifine). After 30 min, the aliquots were observed by a confocal laser scanning microscope (CLSM, Nikon Eclipse Ti, Japan). A WSP5 fluorescent H₂S probe was used for quantifying bacterial H₂S levels by GloMax-Multi microplate multimode reader (Promega, USA). The OD values obtained from different cultures were normalized.

Bactericidal activity against planktonic cells. S. aureus Newman, MRSA, or MDRSA from an exponentially growing culture was diluted to 5×10⁵ bacteria per mL and treated with PBS, Hb, naftifine, Hb-Naf NP, and Hb-Naf@RBCM NP (200 g mL⁻¹ Hb, 40 g mL⁻¹ naftifine) in MHB media at 37° C. under a condition mimicking the infectious microenvironment of the infected tissues (i.e., pH 6.5 and 150 μM H2O2) for 6 h. Then, 50 μL bacteria sample of each group was added into 450 μL Hank's Balanced Salt Solution (HBSS) with or without neutrophils (1×10⁷ cells per mL) at 37° C. After 1 h incubation, bacterial killing was halted and viable bacteria were counted 24 h post-plating of the appropriate dilutions on MH agar plates.

Bactericidal activity against persisters. Stationary-phase cells of S. aureus were employed as a model for persister cells, as has been shown in prior demonstrations. Brief, cells from frozen stocks were incubated in LB broth at 37° C. with 300 rpm shaking and 80% humidity until an OD600 of 0.3 was reached, then diluted 1:1,000 and further cultured for 16 h in 250 mL flasks under identical conditions. The bacteria cells were then washed three times with PBS and diluted to approximately ˜5×10⁷ cells per mL using the same buffer. To assess the persistence of stationary-phase S. aureus Newman and MRSA, they were treated with 100×MIC ciprofloxacin (30 μg mL⁻¹). For stationary-phase MDRSA, which is resistant to ciprofloxacin, 100×MIC vancomycin (100 μg mL⁻¹) was used for the treatment. The persister suspension (˜5×10⁷ CFU/mL) containing 100×MIC ciprofloxacin or 100×MIC vancomycin were added to a 96-well assay block for incubation at 37° C. with 225 rpm shaking. After a 4-hour incubation, 400 μL samples were extracted and washed once with PBS to eliminate the antibiotic. The samples underwent serial dilution with PBS and were plated on MH agar plates. Following an overnight incubation (˜18 h) at 37° C., the colonies were counted to quantify the number of cells.

S. aureus Newman, MRSA, and MDRSA persisters were diluted to 5×10⁵ bacteria per mL and treated with PBS, Hb, naftifine, Hb-Naf NP, and Hb-Naf@RBCM NP (200 g mL⁻¹ Hb, 40 μg mL⁻¹ naftifine) in MHB at 37° C. under conditions mimicking the microenvironment of the infected tissues (i.e., pH 6.5 and 150 μM H₂O₂) for 6 h. Then, 50 μL bacteria sample of each group was added into 450 μL Hank's Balanced Salt Solution (HBSS) with or without human or murine neutrophils (1×10⁷ cells per mL) at 37° C. After 1-hour incubation, the bacterial killing was stopped and live bacteria were counted 24 h after plating appropriate dilutions on MH agar plates.

Bactericidal activity against biofilms. The quantification of S. aureus biofilm mass was carried out in microtiter plates through crystal violet staining following established protocols. Overnight bacterial cultures were diluted to 1×10⁶ cells per mL in tryptic soy broth (TSB) with 0.2% glucose. Subsequently, 200 μL aliquots were placed in each well of a 96-well plate for incubation at 37° C. for 24 h. After removing planktonic cells, the wells were washed with PBS. Fresh TSB containing PBS, Hb, naftifine, Hb-Naf NP, or Hb-Naf@RBCM NP (200 g mL⁻¹ Hb and 40 g mL⁻¹ naftifine) was added to the plates (200 μL per well) and incubated under conditions mimicking the microenvironment of the infected tissues (i.e., pH 6.5 and 150 M H₂O₂) at 37° C. for 6 h. The media from each well were removed and the plates were washed with PBS. HBSS with or without murine or human neutrophils (1×10⁷ cells per mL) was added into each well of the plate and the bacteria culture was then incubated at 37° C. for another 1 hour. Then the bacterial killing was stopped by 25-fold dilution in ice-cold PBS, followed by washing the well three times using PBS. Each well received 200 μL of 0.1% crystal violet, incubated at 37° C. for 15 minutes. After aspiration and four PBS washes, 200 μL of 30% acetic acid was added for absorbance measurement at 595 nm. For viability assay, the biofilm-encased bacteria were dislodged through a 5-min sonication process for the CFU assay after bacterial killing was stopped. Importantly, sonication did not significantly affect bacterial viability.

Release profile. To assess the S. aureus-triggered drug release profiles of naftifine and hemoglobin from Hb-Naf@RBCM NP, Hb-Naf@RBCM NP (100 mg/mL) was subjected to incubation with different S. aureus strains—Newman, MRSA, or MDRSA (1×10⁸ CFU/mL) in 5% (v/v) TSB at 37° C. for 24 h, respectively. Thereafter, the released naftifine and hemoglobin were separated through ultrafiltration using a centrifugal filter unit (MWCO=100 kDa) for 20 minutes at 19,480 g. Quantification of naftifine and hemoglobin was performed using HPLC.

Scanning electron microscopy (SEM). Exponentially growing bacteria (MRSA) were collected by centrifugation at 7,155 g for 5 min, washed with PBS, and diluted to an OD 600 value of 0.5. The bacterial suspensions (1 mL) were incubated with PBS, Hb, naftifine, Hb-Naf NP, or Hb-Naf@RBCM NP with equivalent content of Hb at 200 g mL-1 and naftifine at 40 g mL-1 under conditions mimicking the microenvironment of the infected tissues (i.e., pH 6.5, 150 M H₂O₂) at 37° C. for 2 h. Subsequently, the samples were fixed with 2.5% glutaraldehyde at 4° C. overnight. The fixed samples were washed with PBS (1 mL) followed by MilliQ water (1 mL), and then dehydrated using a series of graded ethanol solutions (1 mL, 50%, 75%, 90%, and 100% for 5 minutes each). After one day of drying, the samples were coated with platinum and observed using SEM with Zeiss/LEO 1530.

Reactive oxygen species (ROS) measurement. Exponentially growing bacteria (MRSA) were collected by centrifugation at 7,155 g for 5 min, washed with PBS, and diluted to an OD 600 value of 0.5. The bacterial suspensions in Eppendorf tubes were then treated with PBS, Hb, naftifine, Hb-Naf NP, or Hb-Naf@RBCM NP with equivalent content of Hb at 200 kg mL⁻¹ and naftifine at 40 g mL⁻¹ and incubated at 37° C. for 2 h. Following incubation, the bacteria were washed with PBS and labeled with a cell-permeable fluorescent dye, carboxy-H2DCFDA (Invitrogen), which was dissolved in DMSO and added to the liquid cultures at a final concentration of 10 μM. The stained bacteria were then examined and visualized under CLSM. The mean fluorescence intensity was assessed by using the Nikon Elements software.

Malondialdehyde (MDA) measurement. Overnight (˜18 h) bacteria cultures of S. aureus Newman, MRSA, or MDRSA were diluted 1:250 in MHB in 250 mL baffled flasks. The cells were grown until they reached an OD600 of 0.2 to 0.3 and were subjected to PBS, Hb, naftifine, Hb-Naf NP, or Hb-Naf@RBCM NP treatments with equivalent content of Hb at 200 g mL⁻¹ and naftifine at 40 g mL⁻¹, under conditions mimicking the microenvironment of the infected tissues (i.e., pH 6.5 and 150 μM H₂O₂) at 37° C. for 2 h. After this time, the cells were collected and centrifuged at 1,748 g for 10 min. The resulting pellets were washed in PBS and centrifuged again at 19,480 g for 5 min. The pellets were then stored at −80° C. Upon thawing, ˜200 μL of B-PER II, containing 100 μg mL⁻¹ of lysozyme and 5 U mL⁻¹ of DNase I, was added to each sample pellet. The samples were then vortexed for 15 min at room temperature and then spun down at 19,480 g for 5 min. The supernatant was frozen at −80° C. until further analysis. The protein concentration of the samples was determined using a BCA assay, while the MDA level was measured using an MDA assay kit (competitive ELISA, ab238537, Abcam).

Lipid peroxides (LPO) measurement. To assay LPO in MRSA, bacteria in exponential growth were pelleted at 8,000 g for 5 min, washed with PBS, and resuspended to an OD600 of 0.5. The bacterial suspensions were incubated with PBS, Hb, naftifine, Hb-Naf NP, or Hb-Naf@RBCM NP with an equivalent content of Hb at 200 g mL⁻¹ and naftifine at 40 g mL-1 under conditions mimicking the microenvironment of the infected tissues (i.e., pH 6.5 and 150 M H₂O₂) at 37° C. for 2 h, followed by washing with PBS. Lipid peroxidation in stained bacteria was monitored with CLSM, using C11-BODIPY 581/591 (Invitrogen D3861, 10 PM) which undergoes a fluorescence shift from red to green upon detection. The mean fluorescence intensity per bacteria was assessed by using the Nikon Elements software.

Transwell chemotaxis assay. Cytoplasmic membranes of S. aureus Newman, MRSA, or MDRSA were isolated from lysostaphin-induced protoplasts as previously described. Briefly, the bacterial cells were washed and resuspended in buffered hypertonic sucrose, followed by treatment with lysostaphin to digest the cell walls. The resulting protoplasts were recovered and lysed in the dilute buffer to extract cytoplasmic membranes. To evaluate the chemotaxis ability of human or murine neutrophils towards S. aureus membranes, a 96-transwell plate with 3 μm pores was used (Corning, USA). The bottom wells were loaded with 200 μL of the bacterial cell membrane in chemotaxis buffer (49% HBSS, 0.5% human serum albumin, 4 mM L-glutamine, and 49% RPMI 1640), while the top wells contained 2×10⁵ neutrophils in 75 μL chemotaxis buffer. The plate was incubated in the dark at 37° C. with 5% CO₂ for 1.5 h. Neutrophils that had migrated into the bottom wells were lysed with elastase assay buffer to release the neutrophil elastase. The activity of elastase was measured using the chromogenic substrate, N-methoxysuccinyl-Ala-Ala-Pro-Val-p-nitroanilide, at a final concentration of 1 mM. The absorbance at 405 nm was measured using a GloMax-Multi microplate multimode reader (Promega, WI, USA) to determine the number of migrated neutrophils.

Measurement of the oxygen release profiles. To assess the oxygen-carrying capacity of Hb, BSA-Naf@RBCM NP, and Hb-Naf@RBCM NP, a dissolved oxygen meter was utilized. The procedure involved purging 1.8 mL of PBS with nitrogen for 10 min to eliminate any dissolved oxygen. The oxygen probe of the dissolved oxygen meter was then immersed in the deoxy PBS solution and the solution was quickly sealed with liquid paraffin. 8 mg of Hb and an equal amount of Hb-Naf@RBCM NP as well as BSA-Naf@RBCM NP dispersed in 200 μL of PBS were subjected to oxygen saturation. The oxygen-saturated samples were slowly injected into the deoxygenated PBS solution sealed with liquid paraffin, and the concentration of dissolved oxygen was recorded with time.

Intracellular hypoxia assay. ROS-ID™ hypoxia/oxidative stress detection kit (Enzo, USA) was utilized to evaluate intracellular hypoxia. Initially, a density of 1×10⁵ 3T3 cells was pre-seeded into the CLSM culture dishes and allowed to incubate for 18 h under normoxia. The cells were then exposed to hypoxic conditions for 6 h and treated with PBS, Hb, naftifine, Hb-Naf NP, or Hb-Naf@RBCM NP for 1 h under the same hypoxic conditions. The hypoxia detection reagent (red) was used to stain the cells, followed by thorough washing with PBS and staining with Hoechst 33342 (blue). Then the cells were examined using CLSM to detect any intracellular hypoxia. The mean fluorescence intensity was assessed by using the Nikon elements software.

Bactericidal activity assay under hypoxic conditions. An exponentially growing culture of S. aureus Newman, MRSA, or MDRSA was diluted with MHB to a concentration of 5×105 per mL and exposed to various treatments, including PBS, Hb, naftifine, Hb-Naf NP, and Hb-Naf@RBCM NP, all containing the same concentration of Hb (200 g mL-1) and Naf (40 g mL-1). The bacteria were then cultured in MHB media conditions mimicking the microenvironment of the infected tissues (i.e., pH 6.5 and 150 μM H₂O₂) and subjected to hypoxic conditions for 6 h. Afterward, 50 μL of each bacteria sample and the respective treatments were added to 450 μL of HBSS with or without human or murine neutrophils (1×107 cells per mL) and incubated under hypoxic conditions at 37° C. for 1 hour. The bacterial killing was stopped and the viable bacteria were counted 24 h post-plating on MH agar plates.

Superoxide anion measurement. To study the superoxide anion generation, neutrophils were cultured in HBSS under hypoxia for 1 h. Following this, various treatments including PBS, Hb, naftifine, Hb-Naf NP, and Hb-Naf@RBCM NP (200 g mL⁻¹ Hb and 40 g mL⁻¹ naftifine) along with 200 nM PMA were added to the cells under hypoxic conditions. After a 30-min incubation period, the superoxide anion assay kit (Sigma Aldrich, USA) was employed to quantify the level of superoxide anion produced.

Intracellular ROS study. To determine the intracellular ROS generation, neutrophils were resuspended at a density of 1×10⁷ cells per mL in either normoxic or hypoxic HBSS and were allowed to incubate under these conditions for 1 h prior to the administration of different treatments, followed by the addition of 1 μM CM-H₂DCFDA for 10 minutes. After this, aliquots of the cell suspensions (225 μL) were added to 25-μL aliquots of zymosan A, and the incubation was allowed to proceed for an hour. The process was terminated by adding BD Cytofix™ Fixation Buffer. The cell suspensions were then diluted in ice-cold PBS and analyzed by flow cytometry to detect ROS production.

In vitro degradation assay. RAW 264.7 murine macrophages were initially seeded into CLSM culture dishes and treated with Cy5-labeled Hb-Naf@RBCM NP for 1 h. Thereafter, the culture media were replaced with fresh ones to remove Cy5-labeled Hb-Naf@RBCM in the media. The nuclei of macrophages were then stained with Hoechst 33342. The same set of macrophages were observed using CLSM at various time points (2 h, 4 h, 6 h, 8 h, and 12 h) post-treatment. The mean fluorescence intensity of Cy5 fluorescence in the captured images was assessed by using the Nikon Elements software.

Cell viability study. Cell viability studies were performed on three different cell lines, including RAW 264.7 macrophages, HEK 293 cells, and NIH 3T3 fibroblasts. After seeding the cells onto a 96-well plate and incubating them overnight, they were treated with various concentrations of Hb-Naf NP and Hb-Naf@RBCM NP. The viability of the cells was then measured using the MTT assay, which relies on the conversion of a tetrazolium salt into formazan crystals by viable cells. The absorbance of the formazan product was measured at 560 nm and 650 nm using a GloMax-Multi Microplate Multimode Reader (Promega, USA) to calculate cell viability.

Animals and ethics. All animal experiments were performed under the Guide for the Care and Use of Laboratory Animals (National Institutes of Health) and protocol (M006549-A03) approved by the Institutional Animal Care and Use Committee (IACUC) at the University of Wisconsin-Madison. ICR mice (22-24 g, female, 8-12-week-old, Charles River) were used for all in vivo experiments. The mice were kept at 22° C. with a 12-h light/dark cycle, 40-50% humidity, and free access to food and water throughout this study.

Mouse thigh infection study. Mice received intramuscular inoculation with MRSA (1×10⁷ CFU per mouse in 50 μL PBS) in their right thigh. Following infection, the mice were randomly assigned to five groups (n=6 biologically independent animals). Two h post-infection, the mice were subjected to treatments via intravenous injection, including PBS, Hb (125 mg kg⁻¹), naftifine (25 mg kg⁻¹), Hb-Naf NP (125 mg kg⁻¹ Hb and 25 mg kg⁻¹ naftifine), and Hb-Naf@RBCM NP (125 mg kg⁻¹ Hb and 25 mg kg⁻¹ naftifine). Twelve h post-infection, mice were euthanized with CO₂, their thigh muscles were harvested, homogenized, and CFUs were evaluated by serial dilution and plating.

For the in vivo neutrophil chemotaxis study, mice received intramuscular inoculation with MRSA (1×10⁷ CFU per mouse in 50 μL PBS) in their right thigh. Following infection, the mice were randomly assigned to three groups (n=3 biologically independent animals). Two h post-infection, the mice were subjected to treatments via intravenous injection, including PBS, Hb-Naf NP (125 mg kg⁻¹ Hb and 25 mg kg⁻¹ naftifine), and Hb-Naf@RBCM NP (125 mg kg⁻¹ Hb and 25 mg kg⁻¹ naftifine). Cy5 was used to label Hb prior to the preparation of Hb-Naf NP and Hb-Naf@RBCM NP in this study. Briefly, Hb (6.45 mg, 100 nmol) and sulfo-Cy5-NHS ester (0.78 mg, 1 mol) were dissolved in a sodium bicarbonate buffer (6.45 mL, pH 8) and stirred at room temperature overnight under nitrogen atmosphere. The Cy5-labeled Hb was then purified via ultrafiltration (molecular weight cut-off=10 kDa). Six h post-infection, mice were euthanized with CO₂ and their quadriceps collected. For immunostaining, the muscles were flash-frozen in liquid nitrogen-cooled isopentane and sectioned at 10 μm using a cryostat. The sections were then immunostained following established protocols. After fixation in 4% paraformaldehyde for 20 min, the slides containing the sections underwent incubation with blocking buffer (PBS added with 10% goat serum) for 1 h before exposure to primary antibodies. The primary antibodies were all employed at a 1:200 dilution for immunostaining. Specifically, anti-Ly6G (Abcam, ab25377) was utilized for immunostaining muscle-infiltrating neutrophils, while anti-laminin (Abcam, ab7463) was employed to delineate the myofiber boundaries. Nuclei were visualized using DAPI (4′,6-diamidino-2-phenylindole). Neutrophil infiltration was quantified by adjusting image thresholds and calculating their percentage area in muscle fields using Nikon Elements software, which also assessed Cy5 fluorescence intensity.

In vivo biodistribution study. To investigate the distribution of the NPs, Mice (n=3 biologically independent animals per group) were infected with MRSA via intramuscular injection (5×10⁶ CFU per mouse in 0.1 mL PBS) one day before imaging. Cy5.5 was used to label Hb before the preparation of Hb-Naf NP and Hb-Naf@RBCM NP in this study. Briefly, Hb (6.45 mg, 100 nmol) and sulfo-Cy5.5-NHS ester (1.11 mg, 1 mol) were dissolved in a sodium bicarbonate buffer (6.45 mL, pH 8) and stirred at room temperature overnight under nitrogen atmosphere. The Cy5.5-labeled Hb was then purified via ultrafiltration (molecular weight cut-off=7 kDa). The Cy5.5-labeled NPs were administered via intravenous (i.v.) injection at 0.5 mg kg-112 h post-infection. The mice were sacrificed at 18 and 36 h post-infection, their major organs (heart, lung, kidneys, liver, spleen, and thighs) were excised, washed with 0.9% NaCl saline, and imaged ex vivo by IVIS. The total influx was quantified by using Live Image 3.2 Software.

Mouse lung infection model study. To evaluate the progression of pathological lesions induced by S. aureus in a mouse pneumonia model, MRSA was administered to mice via intranasal inoculation (5×10⁸ CFU per mouse in 50 μL PBS, with 25 μL per nostril). At various time intervals (5 min, 1 h, 2 h, and 6 h) following infection, the lungs of the infected mice were harvested and subjected to H&E staining for histopathological analysis.

The therapeutical efficacy of Hb-Naf@RBCM NP was evaluated by infecting mice with MRSA by intranasal administration (5×10⁸ CFU per mouse in 50 μL PBS, with 25 μL per nostril). One hour after infection, the mice were randomly divided into five groups (n=6 biologically independent animals per group) and treated with PBS, Hb (125 mg kg⁻¹), naftifine (25 mg kg⁻¹), Hb-Naf NP (125 mg kg⁻¹ Hb and 25 mg kg⁻¹ naftifine), and Hb-Naf@RBCM NP (125 mg kg⁻¹ Hb and 25 mg kg⁻¹ naftifine) via i.v. injection, respectively. Twelve h post-infection, mice were euthanized and lungs were collected, homogenized, and assayed for S. aureus CFU.

Mouse peritonitis model study. Mice were inoculated with MRSA (1×10⁸ CFU each mouse in 0.1 mL PBS) via intraperitoneal (i.p.) injection. The infected mice were then randomly divided into 5 groups (n=6 biologically independent animals per group). One hour after infection, the mice were treated with PBS, Hb (125 mg kg⁻¹), naftifine (25 mg kg⁻¹), Hb-Naf NP (125 mg kg⁻¹ Hb and 25 mg kg⁻¹ naftifine), and Hb-Naf@RBCM NP (125 mg kg⁻¹ Hb and 25 mg kg⁻¹ naftifine) via i.v. injection, respectively. Twelve h post-inoculation, mice underwent CO₂ euthanasia; heart, liver, spleen, lung, kidneys, and ascites were harvested. Ascites were obtained by intraperitoneal PBS injection, abdominal massage, and syringe extraction. Organ and tissue homogenates were processed for CFU determination via serial dilution and plating

For the survival studies, mice were infected with MRSA (1×10⁸ CFU per mouse in 0.1 mL PBS) through i.p. injection on Day 0. Six h after infection, the infected mice were randomly divided into 5 groups (n=8 biologically independent animals per group) and treated with PBS, Hb (125 mg kg⁻¹), naftifine (25 mg kg⁻¹), Hb-Naf NP (125 mg kg⁻¹ Hb and 25 mg kg⁻¹ naftifine), and Hb-Naf@RBCM NP (125 mg kg⁻¹ Hb and 25 mg kg⁻¹ naftifine) via i.v. injection, respectively. Their survival and body weights were recorded every 12 h for 14 days.

Mouse bacteremia model study. Mice were infected with MDRSA (1×10⁸ CFU per mouse in 0.1 mL PBS) by i.v. injection, and the infected mice were randomly separated into 5 groups (n=6 biologically independent animals per group). One hour after infection, the mice were treated with PBS, Hb (125 mg kg⁻¹), naftifine (25 mg kg⁻¹), Hb-Naf NP (125 mg kg⁻¹ Hb and 25 mg kg⁻¹ naftifine), and Hb-Naf@RBCM NP (125 mg kg⁻¹ Hb and 25 mg kg⁻¹ naftifine) via i.v. injection, respectively. Twelve h post-inoculation, the mice underwent CO₂ euthanasia and their major organs (i.e., spleen, lung, kidneys, liver, and heart) and blood were collected. CFUs were determined by homogenizing organs and tissues and plating serial dilutions.

For the survival studies, mice were infected with MDRSA (1×10⁸ CFU per mouse in 0.1 mL PBS) via i.v. injection. Six h after infection, the mice were randomly divided into 5 groups (n=8 biologically independent animals per group) and treated with PBS, Hb (125 mg kg-1), naftifine (25 mg kg⁻¹), Hb-Naf NP (125 mg kg⁻¹ Hb and 25 mg kg⁻¹ naftifine), and Hb-Naf@RBCM NP (125 mg kg⁻¹ Hb and 25 mg kg⁻¹ naftifine) via i.v. injection, respectively. Their survival and body weights were monitored every 12 h for 14 days.

Histopathological and safety studies. For studies in the mouse bacteremia model, mice were first i.v. inoculated with MDRSA (1×10⁸ CFU per mouse in 0.1 mL PBS) and then randomly separated into 5 groups (n=3 biologically independent animals per group). One hour after infection, mice received PBS, Hb (125 mg kg⁻¹), naftifine (25 mg kg⁻¹), Hb-Naf NP (125 mg kg⁻¹ Hb and 25 mg kg⁻¹ naftifine), and Hb-Naf@RBCM NP (125 mg kg⁻¹ Hb and 25 mg kg⁻¹ naftifine) via i.v. injection, respectively. Twelve h post-inoculation, the mice were euthanized using CO₂ asphyxiation with their major organs collected.

For studies using healthy mice, mice (n=3 biologically independent animals per group) received a single i.v. injection of PBS, Hb-Naf NP (125 mg kg⁻¹ Hb, 25 mg kg⁻¹ naftifine), or Hb-Naf@RBCM NP (125 mg kg⁻¹ Hb, 25 mg kg⁻¹ naftifine) on Day 0, Day 2 and Day 4. A total of three injections were applied to each mouse. On Day 7, the mice were sacrificed, followed by blood and major organs collection. In addition, to evaluate the proinflammatory cytokine levels in the mouse serum, mice (n=3 biologically independent animals per group) received i.v. injection of PBS and a doubled dose of Hb-Naf@RBCM NP (250 mg kg⁻¹ Hb, 50 mg kg⁻¹ naftifine). Six h after injection, their blood was collected.

Organs were paraffin-embedded, sectioned at 5 m, and stained with hematoxylin and eosin (H&E) for histopathological analysis. Plasma was obtained by centrifuging blood at 1,006 g for 10 minutes at 4° C. Biochemical profiles were analyzed with a VetScan VS2 blood chemistry analyzer (Abaxis). Proinflammatory cytokines IL-6, TNF-α, and IL-1β levels were measured with ELISA kits (R&D Systems, M6000B, MTA00B, and MLB00C).

Statistics and reproducibility. Each experiment, including TEM, confocal microscopy images, and SEM, was independently repeated at least three times. Data were analyzed directly and presented as mean±standard deviation (s.d.). Mice and microscopic fields were randomly assigned to treatments and inspections, respectively. Statistical comparisons between experimental groups were conducted using a one-way ANOVA test, followed by Tukey's post hoc comparison test or a two-tailed Student's t-test. The survival benefit was determined using a log-rank test. A P-value of 0.05 or higher was considered to indicate non-significance at the 95% confidence level. All statistical analyses were carried out using GraphPad Prism version 9.0.

### Example 1: Preparation and Characterization of Hb-Naf@RBCM NP

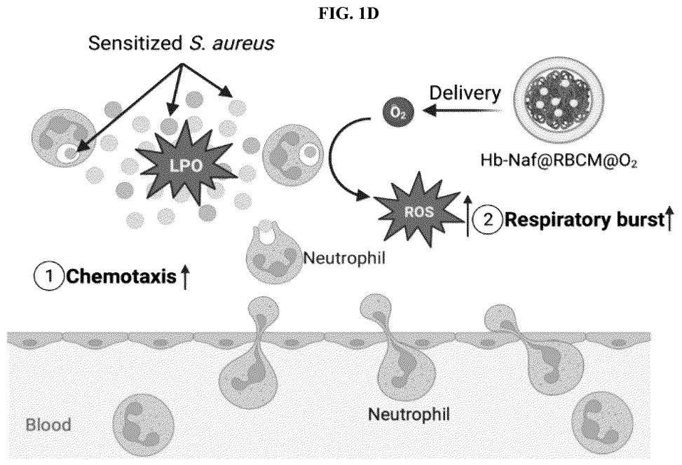

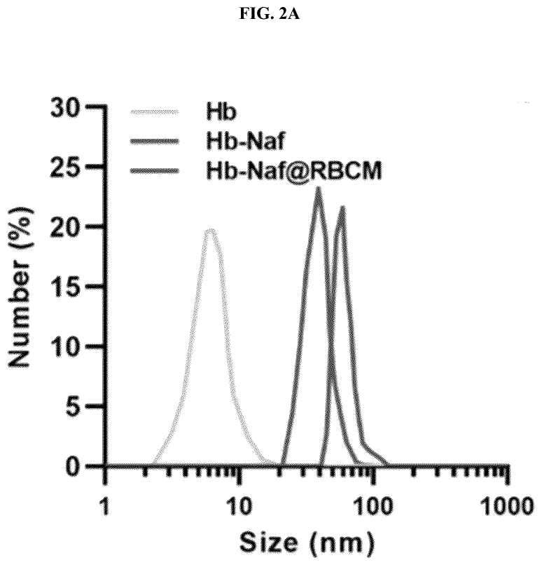

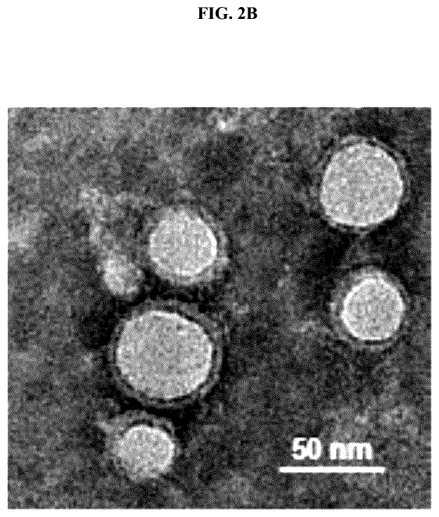

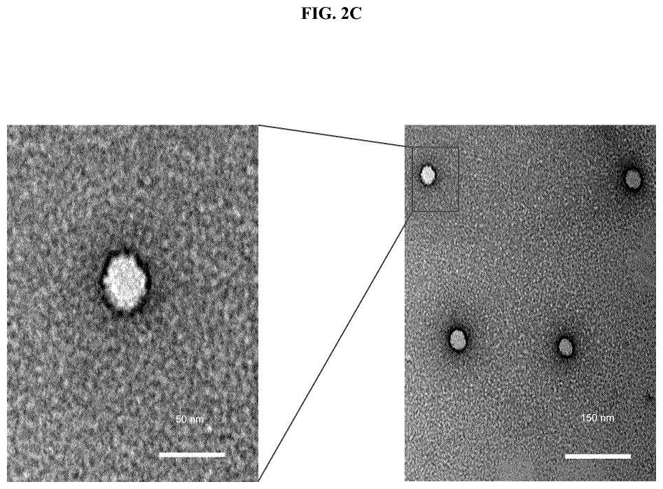

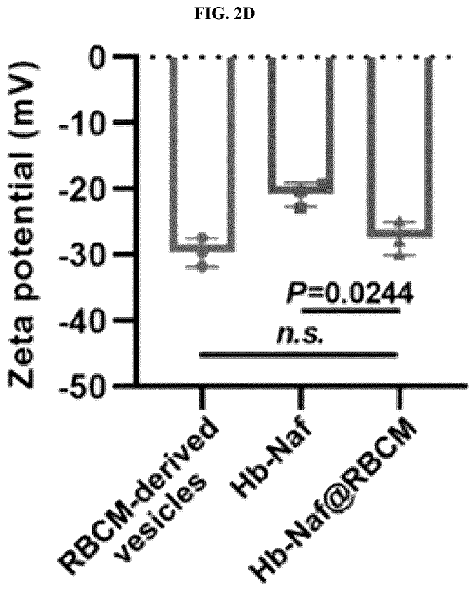

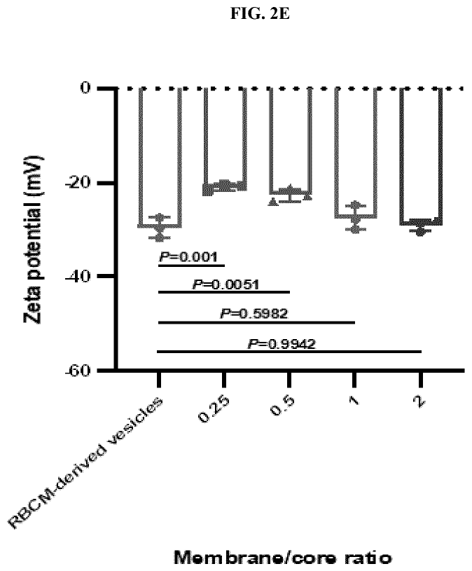

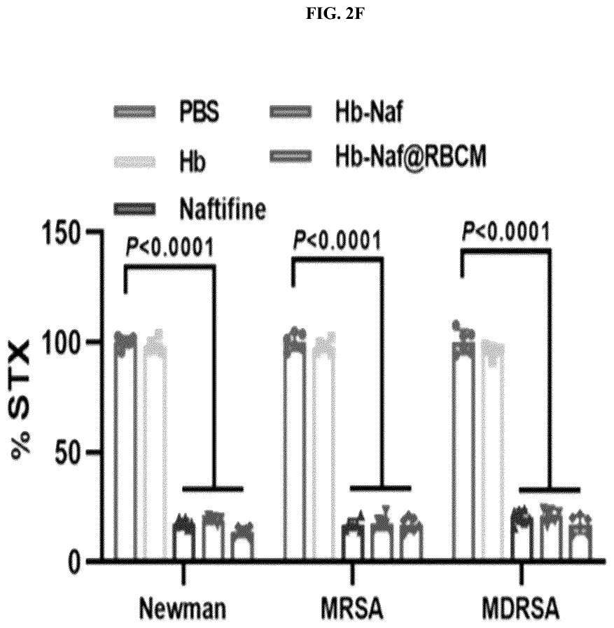

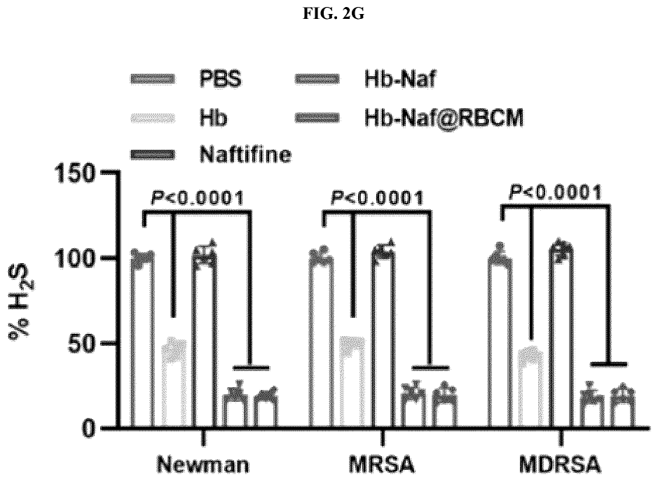

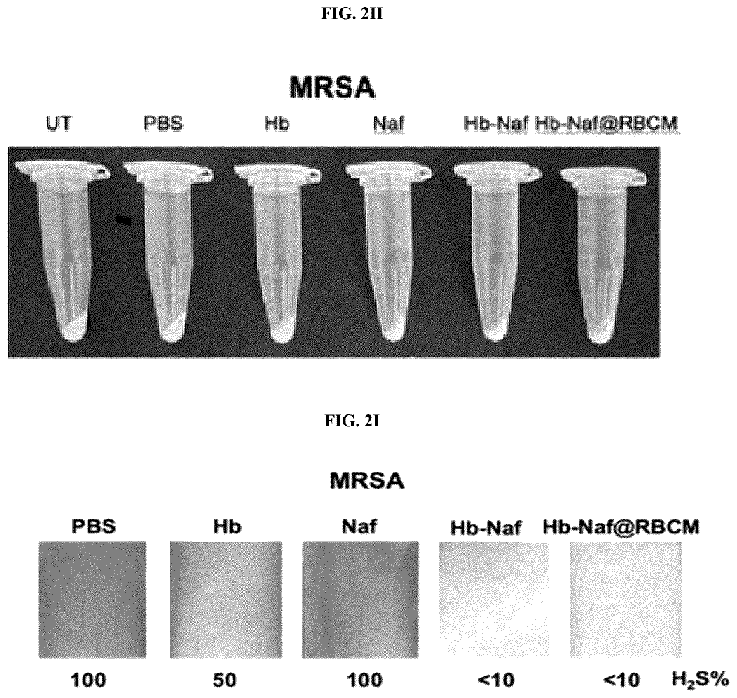

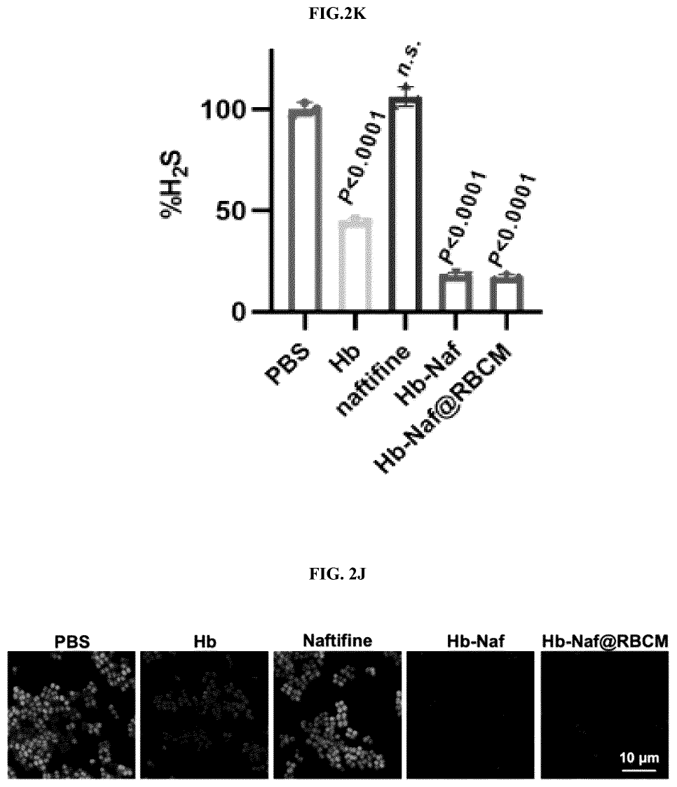

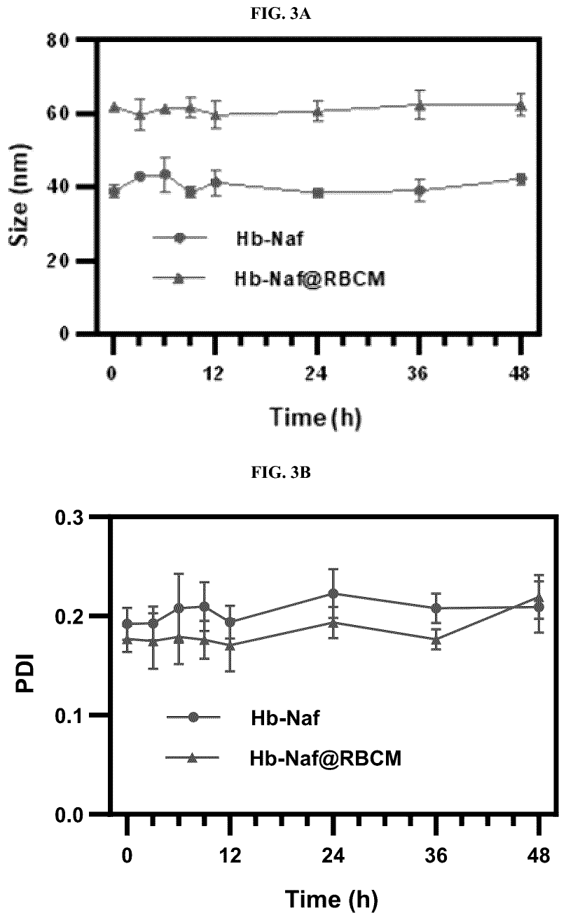

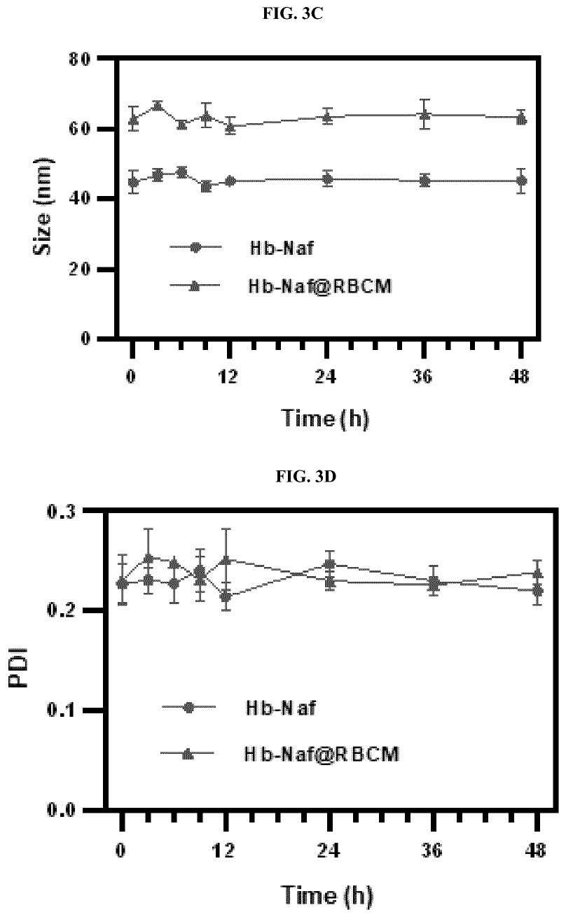

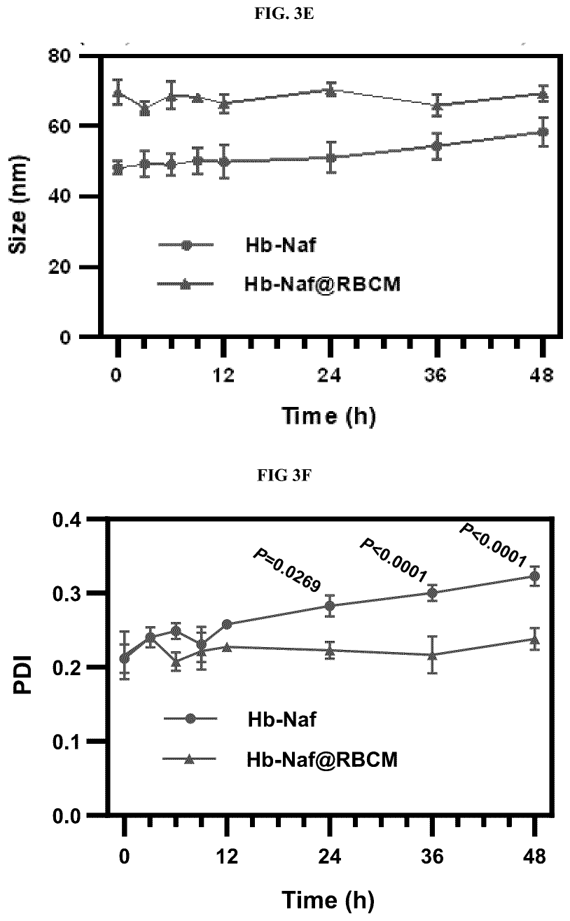

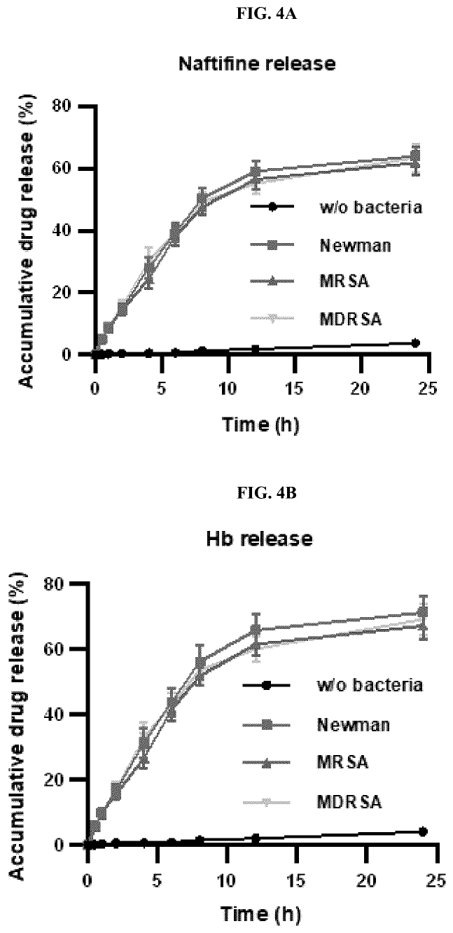

Hb-Naf NP was formed via the self-assembly of naftifine and Hb through hydrophobic interactions. Hb-Naf@RBCM NP was obtained via extrusion of Hb-Naf NP together with RBCM-derived vesicles. Hb-Naf@RBCM NP fabricated with optimized naftifine/Hb and RBCM/Hb-Naf ratios (Table 1) had a hydrodynamic diameter of approximately 62 nm, quantified by dynamic light scattering (DLS, FIG. 2A). In contrast, the size of the uncoated Hb-Naf NP was around 45 nm (FIG. 1e). The diameter of Hb-Naf@RBCM NP under transmission electron microscopy (TEM, FIG. 2B, 2C) was around 50 nm with a distinct 7-nm membrane coating, consistent with the thickness of RBCM reported previously. Hb-Naf@RBCM NP exhibited a zeta potential of −28 mV, similar to that of RBCM-derived vesicle (−29 mV, FIG. 2D, 2E). Collectively, these results demonstrate successful RBCM coating. Hb-Naf@RBCM NP contained 41.5 wt % Hb and 8.2 wt % naftifine, with respective loading efficiencies of 86.3% and 63.5%. It remained stable in various sterile conditions (FIGS. 3A-3H). However, the presence of S. aureus led to the destabilization of Hb-Naf@RBCM NP and the release of 63.0% of naftifine and 69.1% (FIGS. 4A and 4B) of Hb within 24 h, likely because S. aureus toxins lysed RBCM.

TABLE 1

| Naftifine/Hb (mol/mol) | 10/1 | 20/1 | 30/1 | 40/1 | 50/1 |
|---|---|---|---|---|---|
| Size (nm) | 57 ± 4 | 41 ± 3 | 45 ± 4 | 72 ± 5 | 145 ± 7 |
| PDI | 0.224 ± 0.011 | 0.216 ± 0.015 | 0.209 ± 0.018 | 0.247 ± 0.022 | 0.282 ± 0.034 |
| Naftifine loading | 43.1% | 53.3% | 68.7% | 57.6% | 46.3% |
| efficiency | | | | | |

### Example 2: Antimicrobial Hb-Naf@RBCM NP Sensitized S. aureus to Neutrophil Killing

S. aureus employs multiple antioxidant defense mechanisms including STX pigment and H₂S. Hb-Naf@RBCM NP delivers naftifine that inhibits STX production. It also delivers Hb that reduces H₂S levels through metHb-catalyzed oxidation of H₂S to thiosulfate and polysulfides. Both mechanisms counter S. aureus's resistance against oxidative stress. Hb-Naf@RBCM NP successfully reduced STX levels in S. aureus strains including S. aureus Newman (ATCC 25904), MRSA (ATCC 33591), and multidrug-resistant (MDR) S. aureus (MDRSA, ATCC BAA-44), resulting in colorless bacteria due to significant reduction in carotenoid pigment synthesis (FIGS. 2F-2G). Moreover, Hb-Naf@RBCM NP efficiently reduced H₂S levels across all S. aureus strains, confirmed by a lead acetate test (FIG. 2H) and a WSP5 fluorescent H₂S probe (FIG. 2I). MRSA treated with Hb-Naf@RBCM NP showed significantly reduced H₂S levels, as observed through confocal laser scanning microscopy (CLSM) using an H₂S-specific twisted internal charge transfer (TICT)-based fluorescent probe (FIGS. 2J and 2K). Interestingly, compared to Hb alone, naftifine-encapsulated NPs further decreased H₂S levels by reducing STX, an antioxidant that counteracts the Hb-induced oxidation of H₂S.

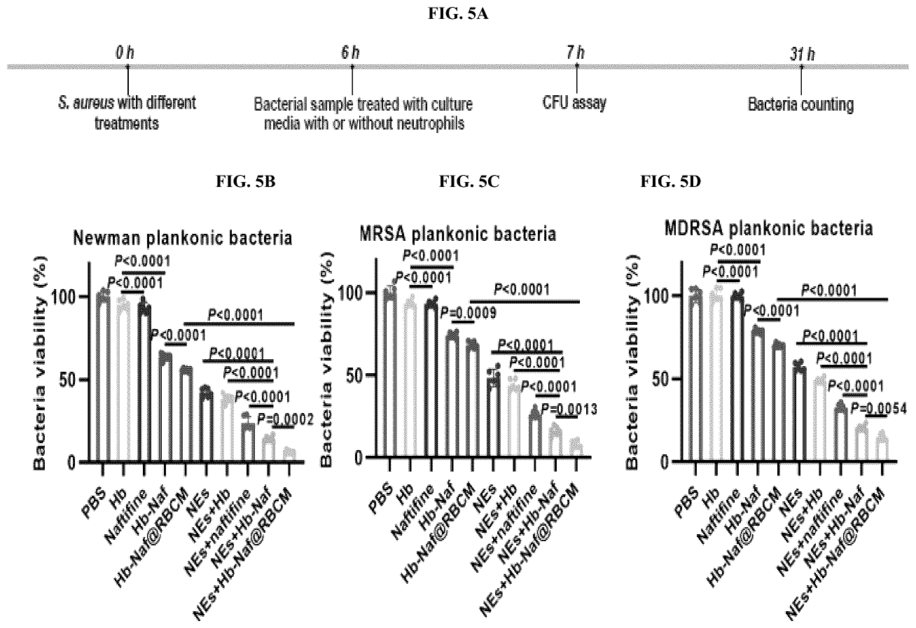

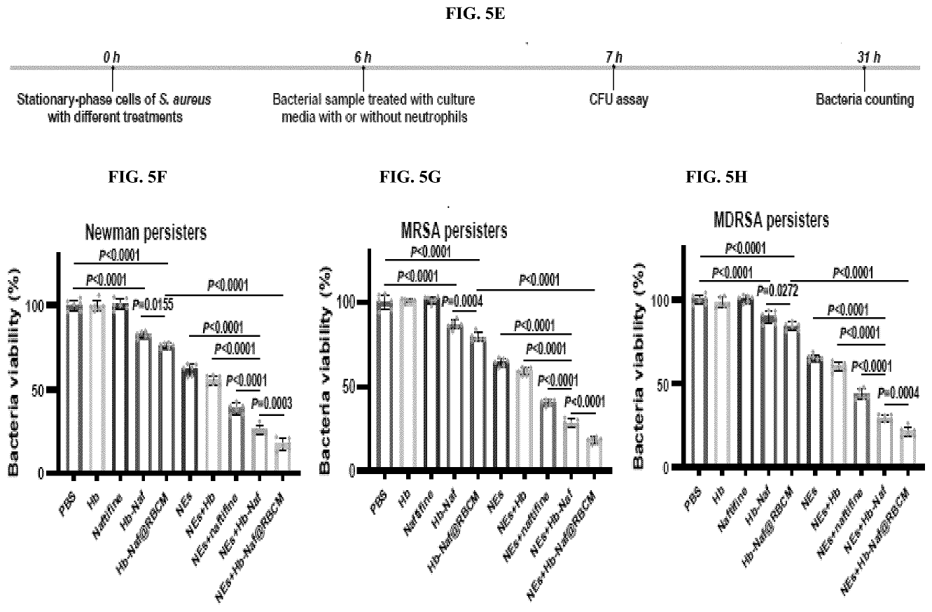

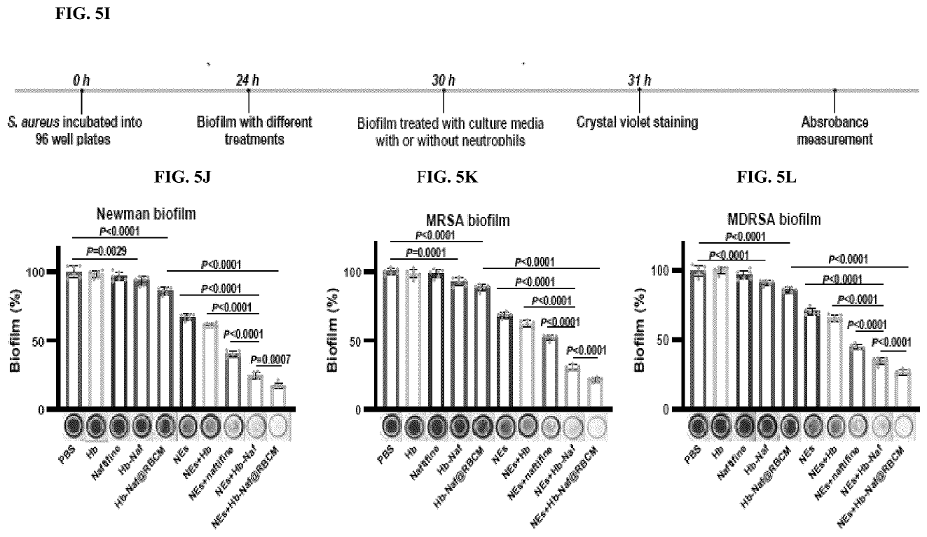

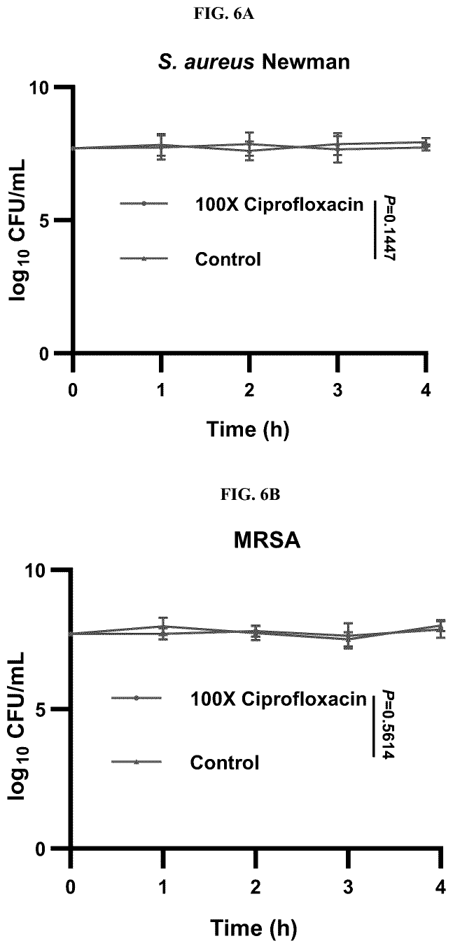

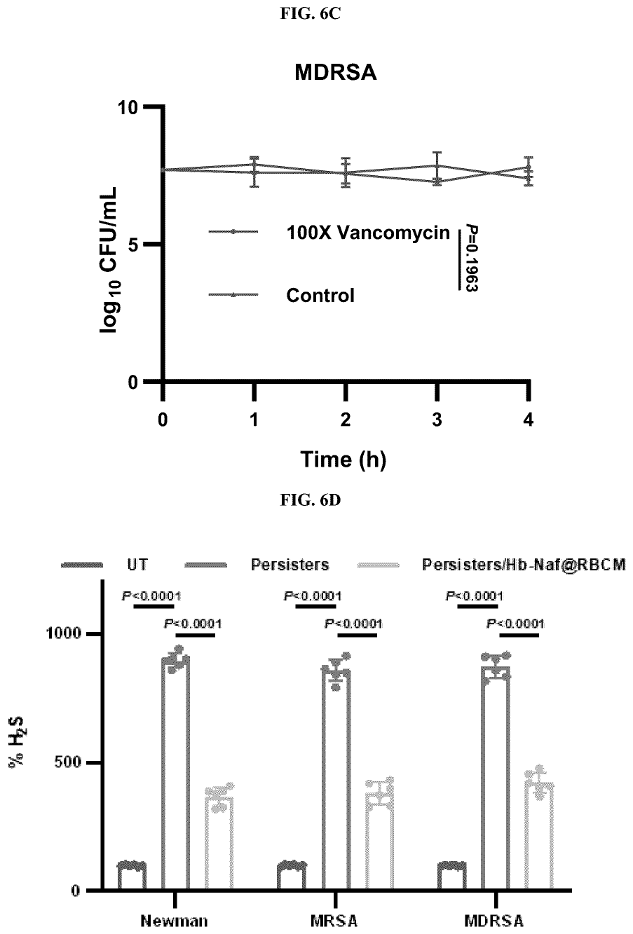

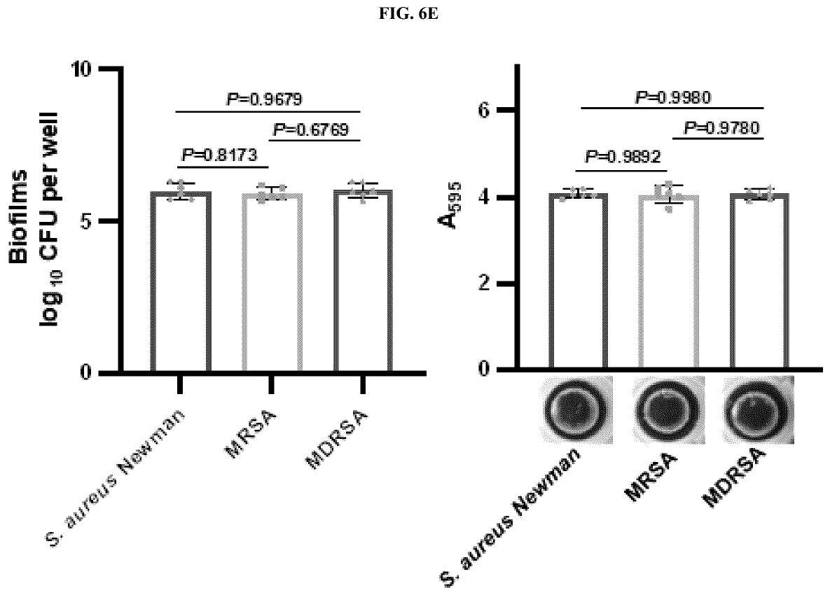

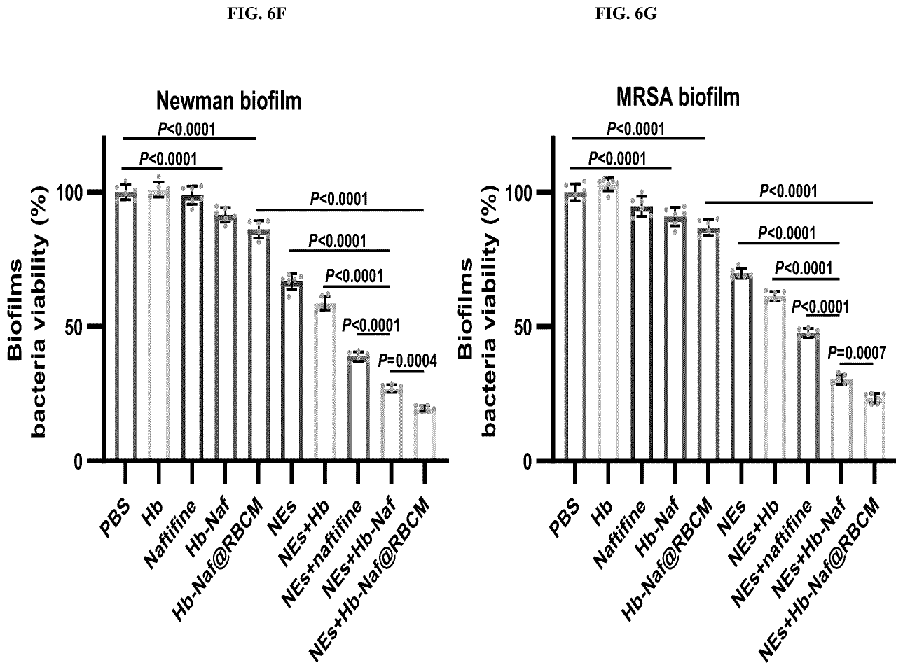

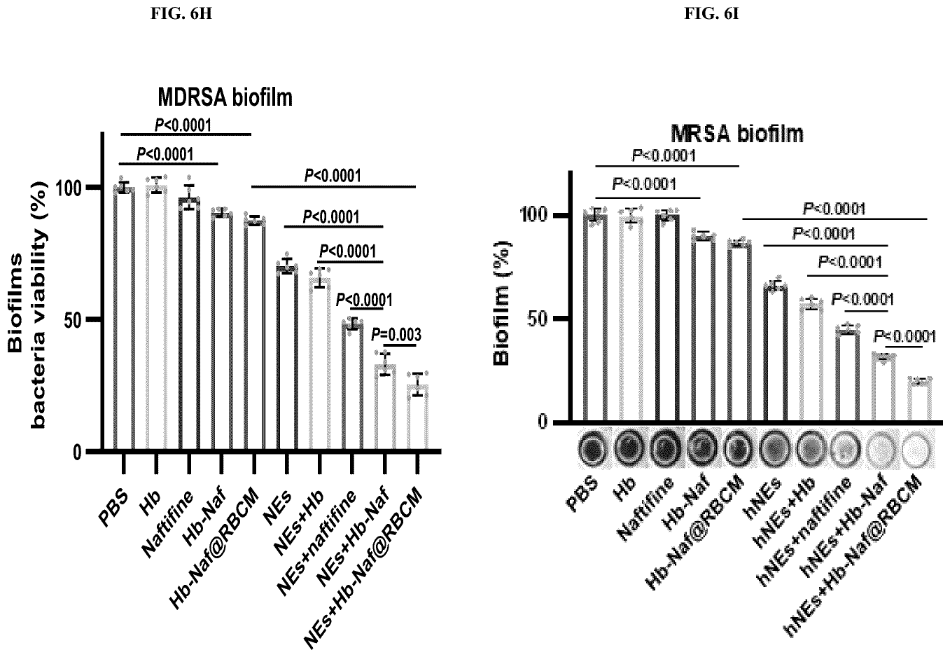

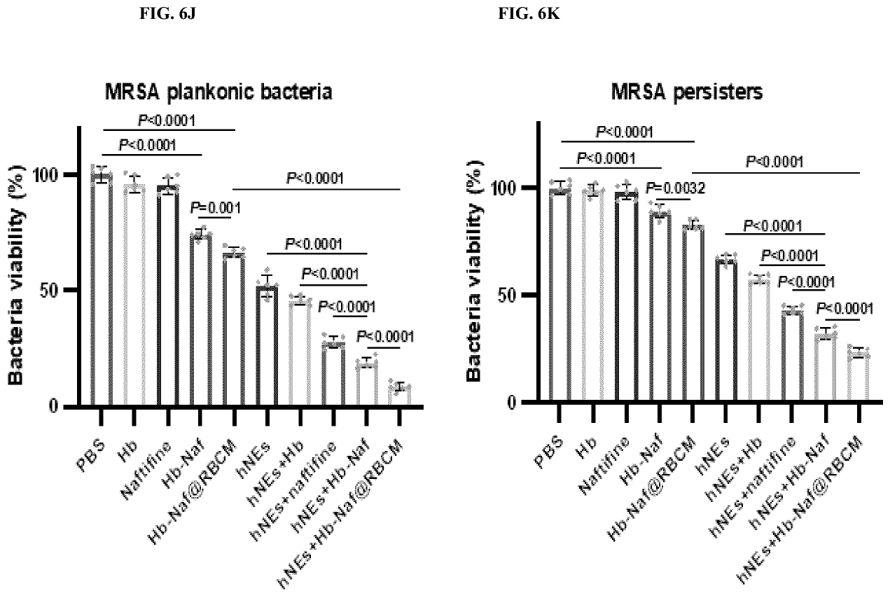

Since Hb-Naf@RBCM NP significantly impaired S. aureus's antioxidant defense systems, we conducted a series of in vitro studies to evaluate Hb-Naf@RBCM NP's effectiveness against S. aureus planktons, persisters, and biofilms under normoxic conditions. (1) Planktons: The combination of neutrophils with Hb-Naf@RBCM NP exhibited superior bactericidal effectiveness against all strains of plankton, unlike individual treatments including Hb-Naf NP, Hb-Naf@RBCM NP, or neutrophils (FIGS. 5A-5D). (2) Persisters: Given S. aureus's ability to survive antibiotic treatments through metabolically slow and highly tolerant persisters, the bactericidal efficacy against S. aureus persisters using stationary-phase cells was studied (FIGS. 6A-6C). While H₂S is known to be critical in establishing and maintaining the persister state, Hb-Naf@RBCM NP significantly decreased the H₂S levels of persisters (FIG. 6D). Combining neutrophils with Hb-Naf@RBCM NP effectively killed antibiotic-resistant persisters (FIGS. 5E-5H). This approach outperformed other treatments, where individual or non-combined strategies led to less than 50% inhibition. (3) Biofilms: Bacterial biofilms, prevalent in healthcare-associated infections and on implantable medical devices, resist antibiotics due to persisters and hypoxia. The efficacies of Hb-Naf@RBCM NP against various biofilms (FIG. 6E) were studied. The combination of neutrophils and Hb-Naf@RBCM NP notably reduced bacterial counts in biofilms of S. aureus Newman, MRSA, and MDRSA and decreased their biofilm mass, outperforming neutrophil treatment alone (FIGS. 6F-6H, FIGS. 5I-5L). We further studied the antimicrobial efficacies of the NPs against MRSA with human neutrophils (FIGS. 6I-6K). Similar to our findings using murine neutrophils, Hb-Naf@RBCM NP exhibited notable antimicrobial efficacy by significantly reducing bacterial burden in MRSA planktons, persisters, and biofilms, respectively, and also diminishing the MRSA biofilm mass.

### Example 3: Hb-Naf@RBCM NP Facilitated Neutrophil Chemotaxis

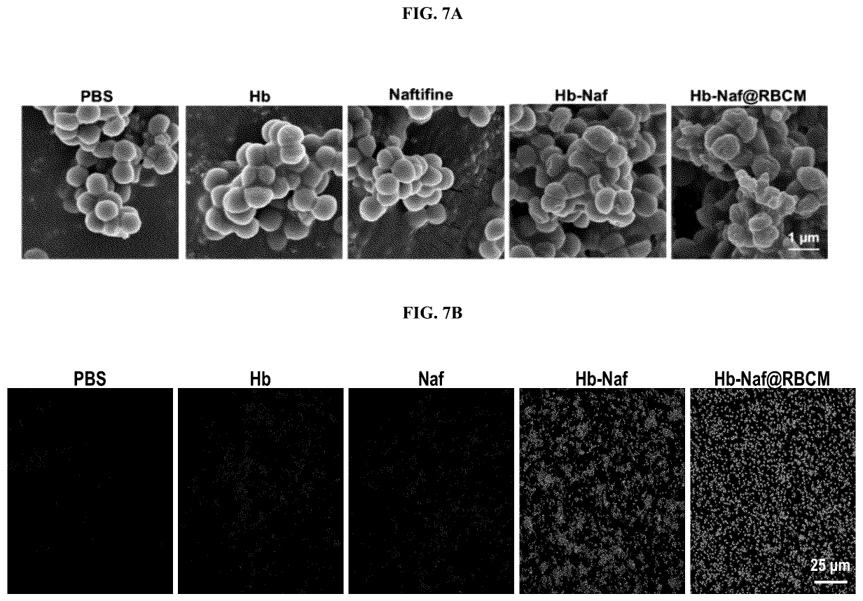

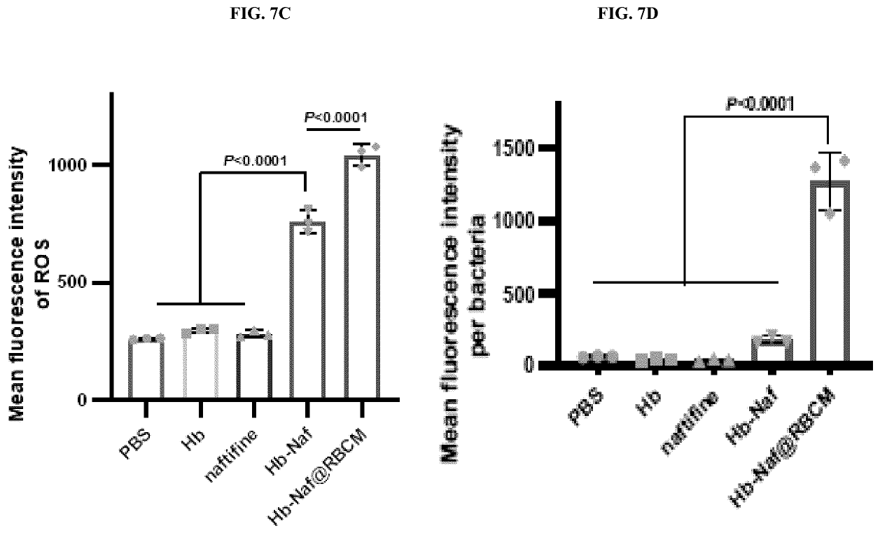

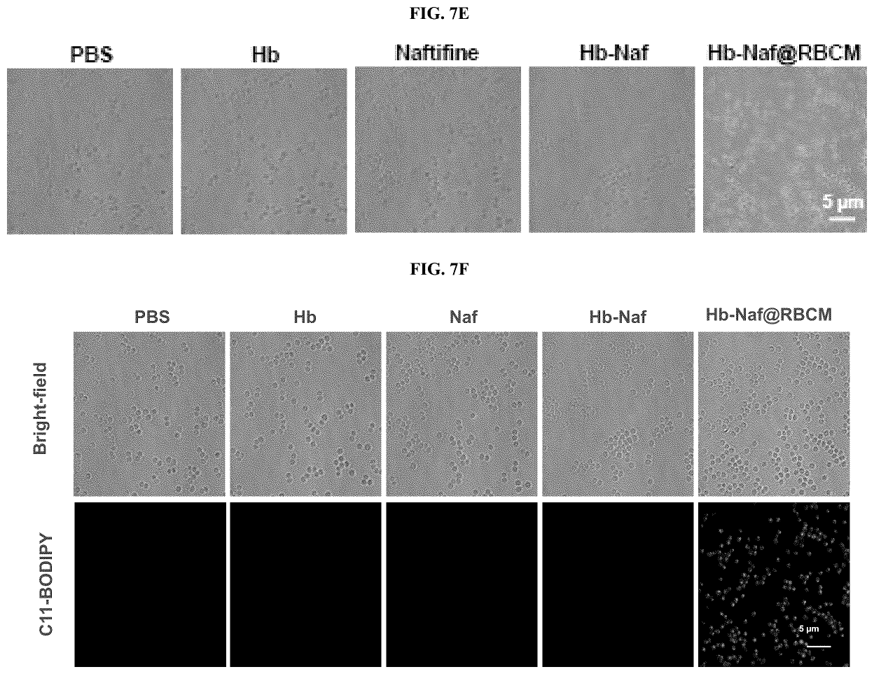

Hb can be transformed into a highly oxidative ferryl form under low pH and high H₂O₂, leading to lipid peroxidation where carbon-carbon double bonds in PUFAs are oxidized. This process results in lipid peroxide accumulation in bacterial membranes, leading to membrane rupture and cell damage. S. aureus cannot synthesize PUFAs, but it can incorporate exogenous PUFAs into its cell membrane via fatty acid phosphorylation. We thus hypothesized that Hb-Naf@RBCM NP can deliver PUFAs into S. aureus and facilitate lipid peroxidation. To investigate this, MRSA cell membrane morphology changes after various treatments were observed under scanning electron microscopy (SEM). The cell membranes of MRSA treated with Hb-Naf@RBCM NP had a higher level of roughness and deformation than those treated with Hb-Naf NP (FIG. 7A). Furthermore, the reactive oxygen species (ROS) levels in MRSA were measured using carboxy-H₂DCFHDA via CLSM. MRSA treated with Hb-Naf@RBCM NP exhibited higher ROS levels than those treated with Hb-Naf NP (FIGS. 7B, 7C), indicating that RBCM contributed to elevated ROS levels.

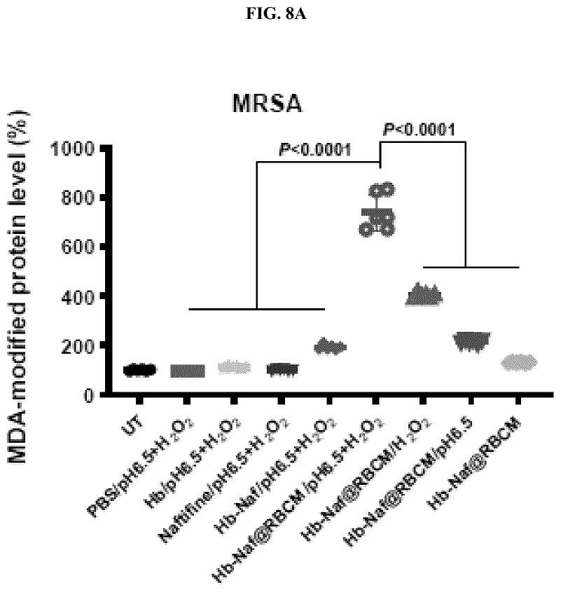

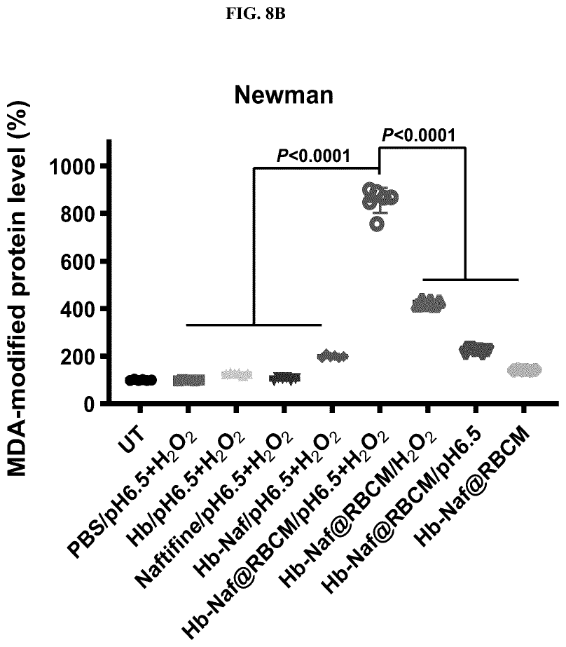

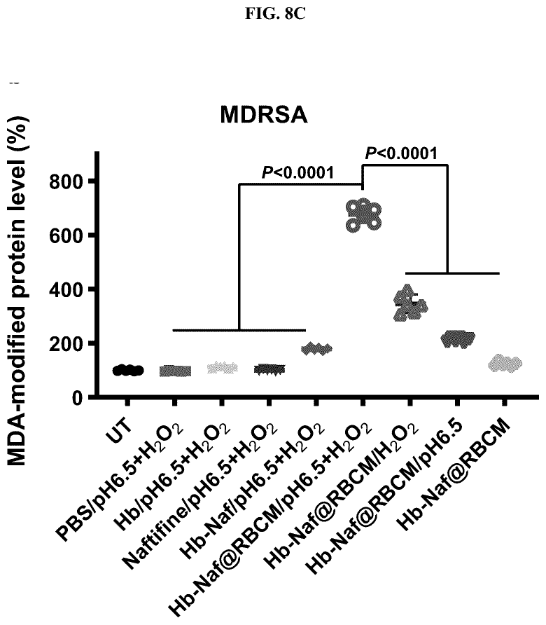

The effects of Hb-Naf@RBCM NP on lipid peroxidation in MRSA were further studied using a C11-BODIPY lipophilic probe and CLSM. MRSA treated with Hb-Naf@RBCM NP exhibited significant lipid peroxidation positive signals in almost all MRSA cells (FIGS. 7D-7F), suggesting the incorporation of PUFAs from the RBCM lysate into the MRSA cell membranes. Moreover, we measured S. aureus's malondialdehyde (MDA)-modified protein levels under conditions mimicking infectious microenvironment (FIGS. 8A-8C). Under conditions mimicking infectious microenvironment, the MDA-modified protein levels of Hb-Naf@RBCM NP-treated MRSA significantly increased compared to the PBS control. Further analysis showed that Hb-Naf@RBCM NP-induced lipid peroxidation was responsive to low pH and high H₂O₂, as Hb induces lipid peroxidation in the presence of H₂O₂, and the process is substantially amplified at pH 6.5. These results demonstrated the combined effects of ferryl Hb and RBCM, which enabled Hb-Naf@RBCM to effectively induce lipid peroxidation in S. aureus.

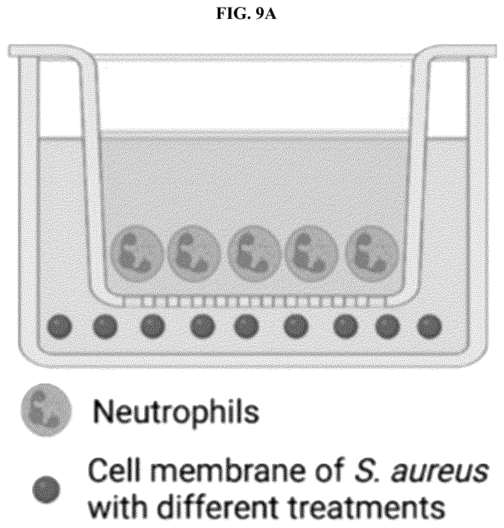

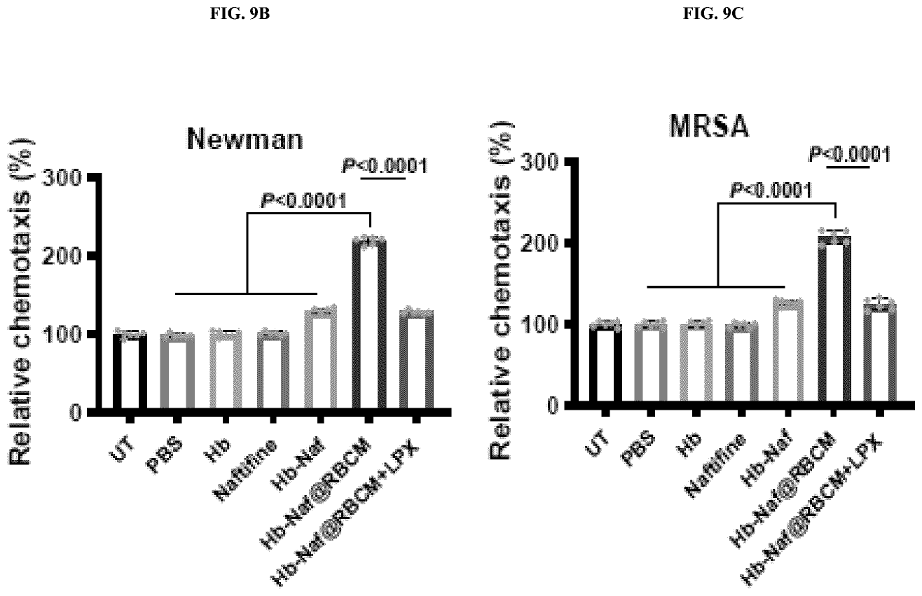

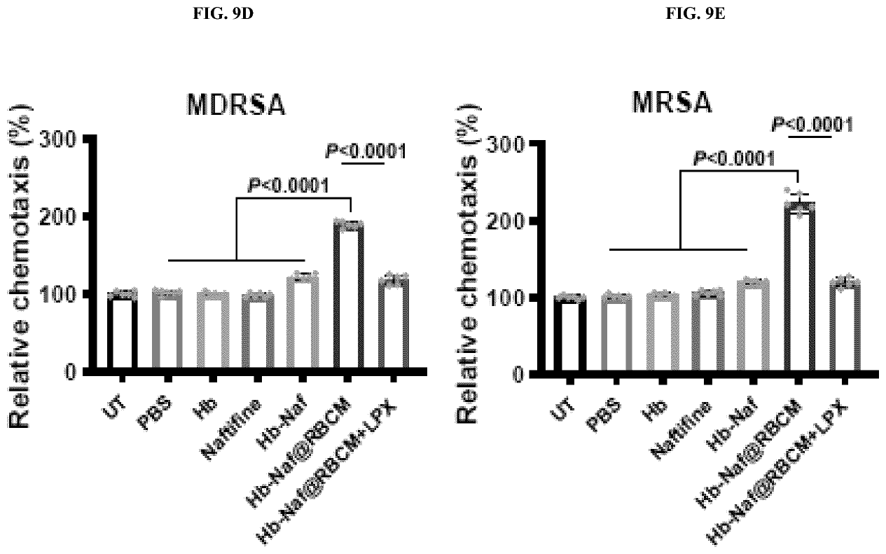

The recruitment of neutrophils is a crucial step in controlling infections. Lipid peroxidation plays an important role in promoting neutrophil chemotaxis. Transwell cell migration assay results showed that Hb-Naf@RBCM NP treatment notably increased the recruitment of murine or human neutrophils to S. aureus cell membranes compared to the PBS control (FIGS. 9A-9E). Liproxstatin-1, a lipid radical scavenger, significantly inhibited the Hb-Naf@RBCM-induced neutrophil recruitment, indicating that this process is lipid peroxidation-dependent.

### Example 4: Hb-Naf@RBCM NP Restored Oxidant Killing Under Hypoxia

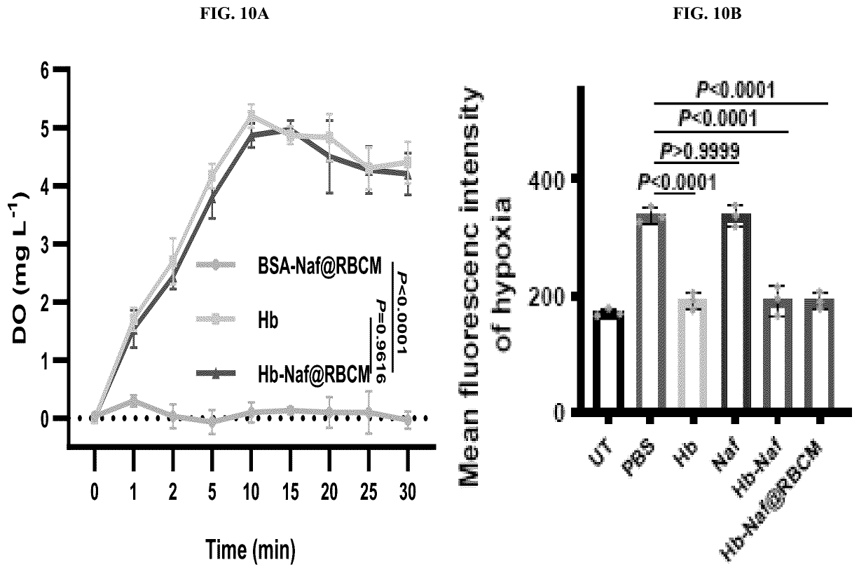

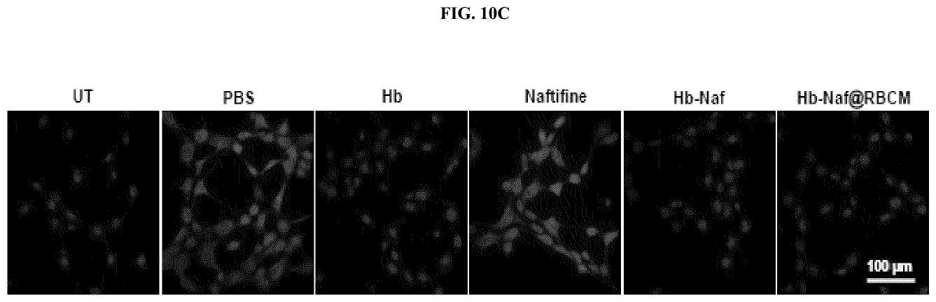

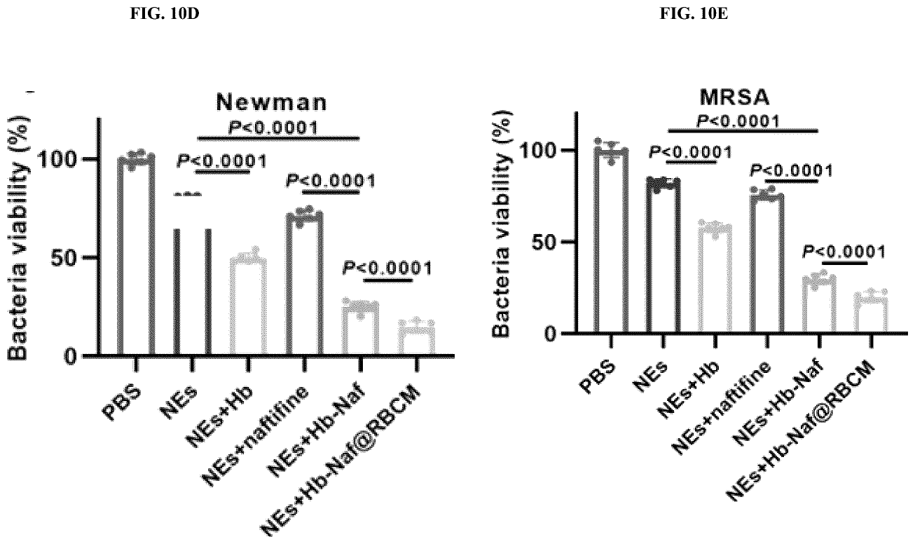

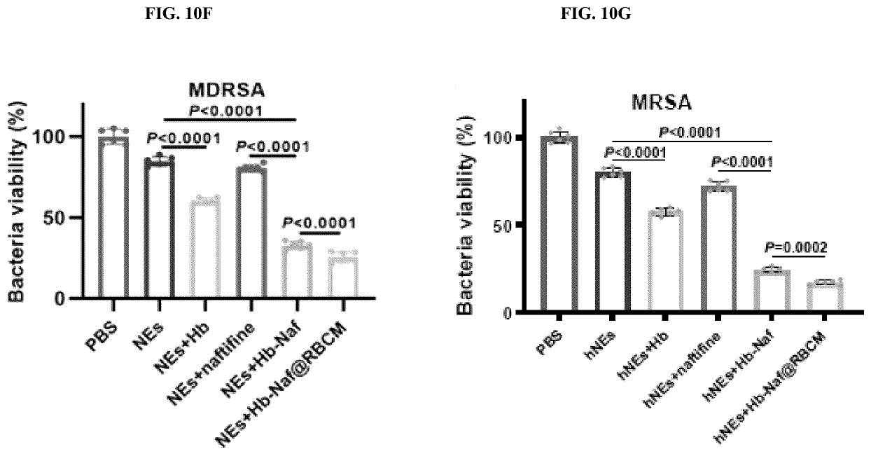

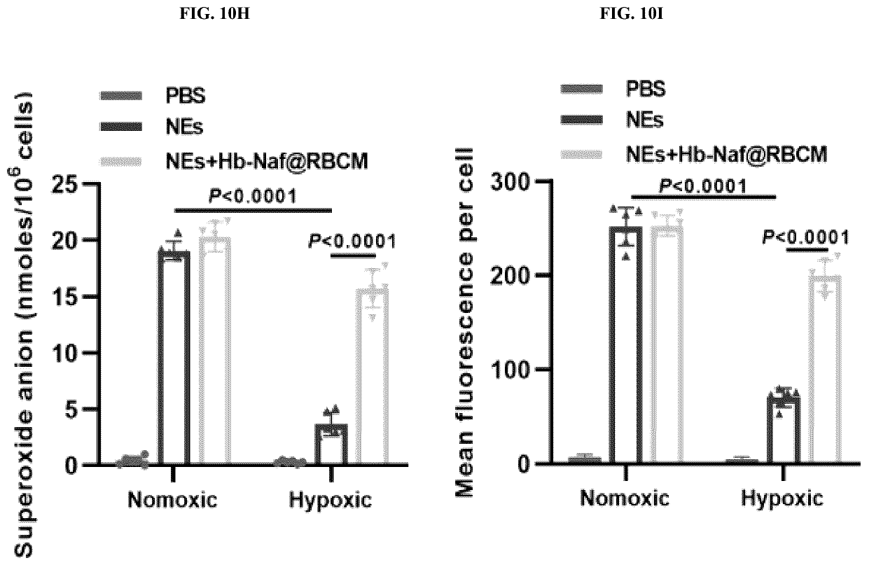

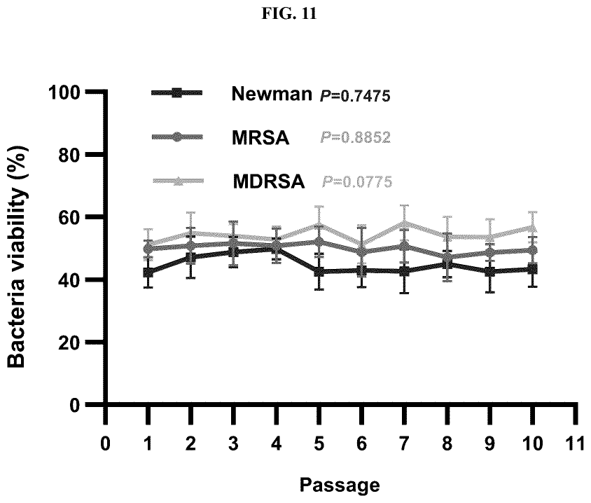

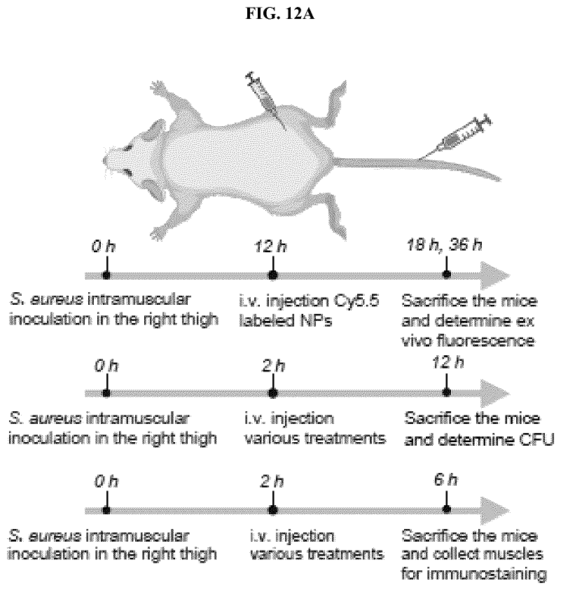

Neutrophil activation plays a crucial role in combating infections through the production of superoxide and other ROS. However, the respiratory burst of neutrophils is significantly impaired in the hypoxic infectious microenvironment for S. aureus clearance. We hypothesized that Hb-Naf@RBCM NP containing oxygenated Hb can deliver oxygen to hypoxic infected tissue and enhance neutrophils' respiratory burst against S. aureus. Our studies confirmed the oxygen-carrying capacity of Hb-Naf@RBCM NP (FIG. 10A). Analyses employing the ROS-ID® hypoxia/oxidative stress detection kit and CLSM showed that Hb-Naf@RBCM NP alleviated cellular hypoxia in NIH 3T3 fibroblasts (FIGS. 10B, 10C). In vitro experiments showed that formulations with Hb greatly enhanced the antimicrobial effectiveness of murine and human neutrophils, with Hb-Naf@RBCM NP showing the highest inhibition rates (FIGS. 10D-10G). Furthermore, Hb-Naf@RBCM NP enhanced neutrophils' production of extracellular superoxide anion and intracellular ROS (FIGS. 10H, 10I). No detectable development of antimicrobial resistance in S. aureus strains to this nanoimmunotherapy was observed under hypoxic conditions after ten passages (FIG. 11).

### Example 5: Hb-Naf@RBCM NP Exhibited Excellent Efficacy and Safety

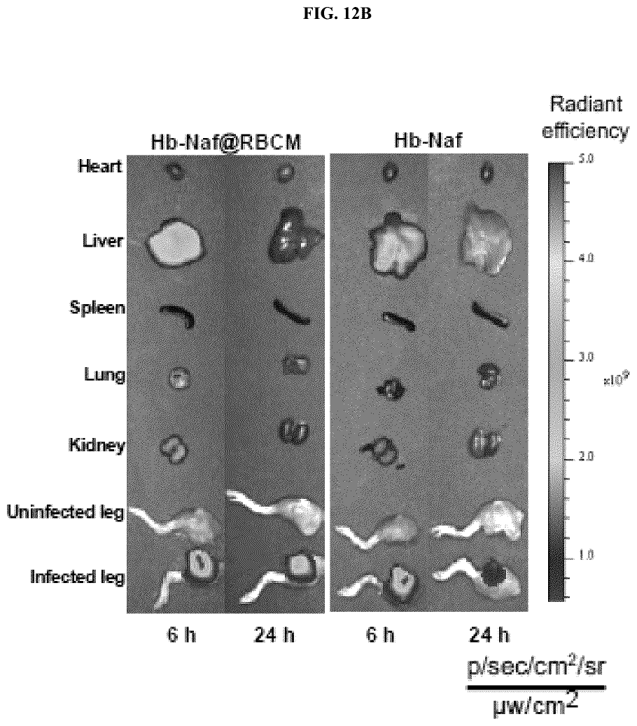

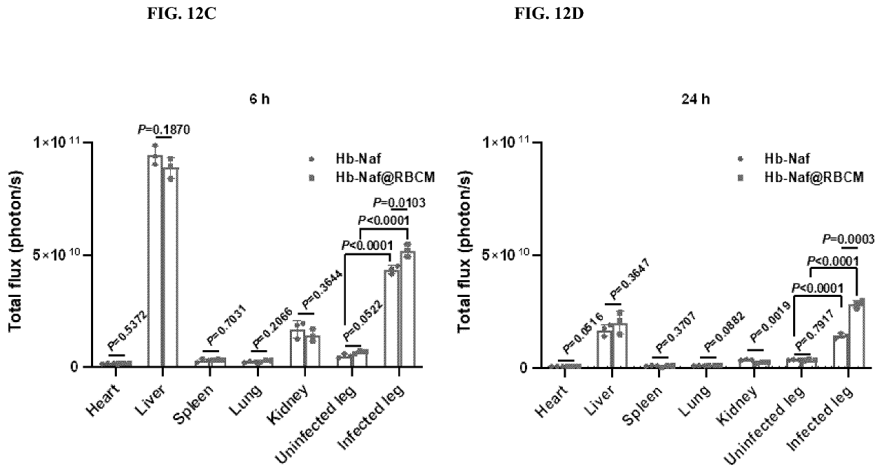

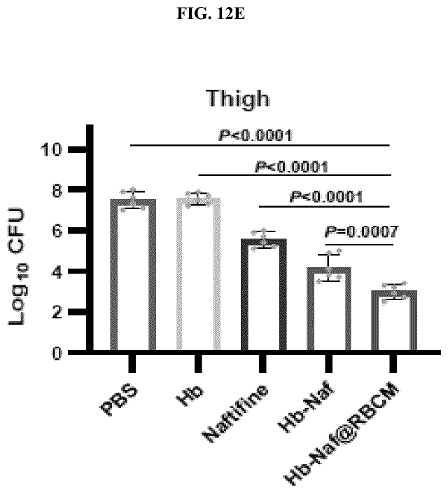

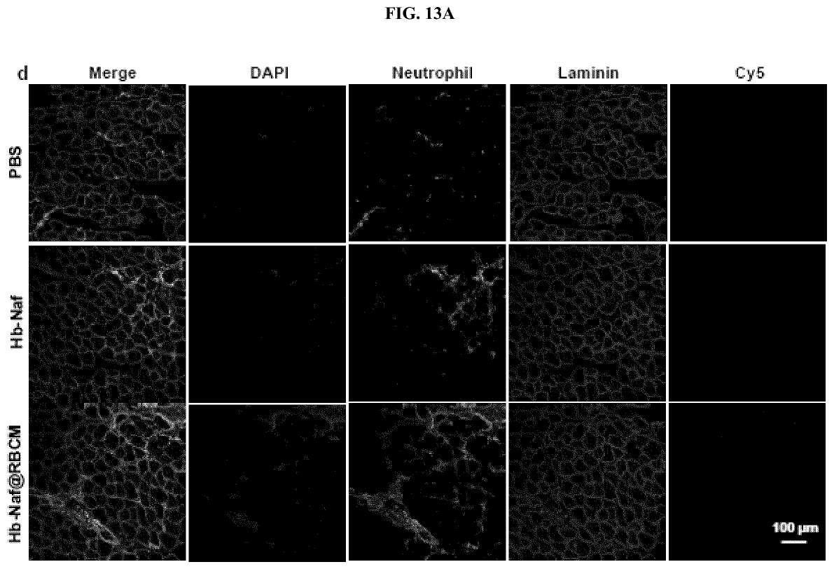

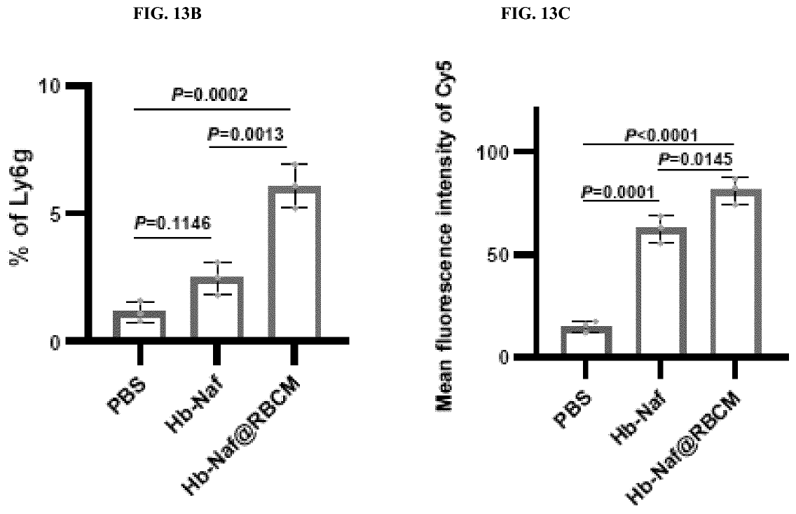

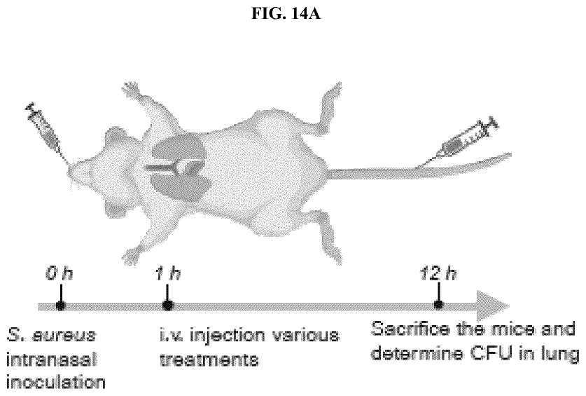

The biodistribution of intravenously (i.v.) injected Cy5.5-labeled Hb-Naf NP and Hb-Naf@RBCM NP in mice with MRSA thigh infections was analyzed using an in vivo imaging system (IVIS, FIG. 12A). At 6 and 24 h after injection, mice showed 7.7-fold and 8.3-fold accumulation of Hb-Naf@RBCM NP in infected thighs than uninfected ones (FIGS. 12B-12D). The preferential accumulation of Hb-Naf@RBCM NPs at infection sites is likely due to the enhanced permeability and retention (EPR) effect, which is further augmented by the RBCM coating that prolongs NP circulation time. Hb-Naf@RBCM NP also effectively reduced the bacterial burden in the infected thigh to 0.0033% of the PBS control (FIG. 12E). Moreover, Hb-Naf@RBCM NP treatment was found to promote neutrophil chemotaxis to the infection site, as indicated by immunostaining results (FIGS. 13A-13C). This supports the hypothesis that Hb-Naf@RBCM NP induces lipid peroxidation in S. aureus to facilitate neutrophil migration to the infection site and oxidant killing of S. aureus.

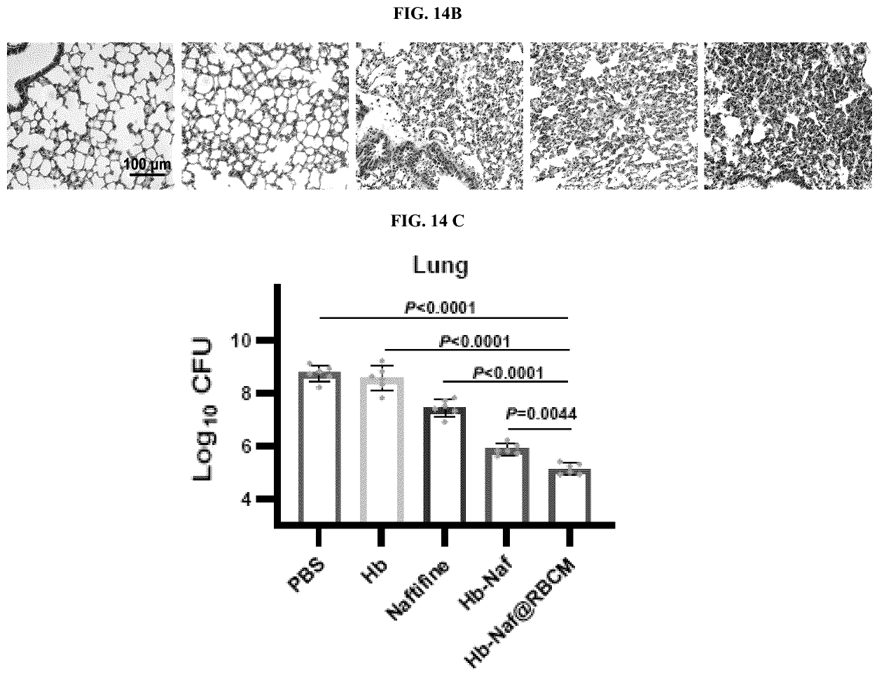

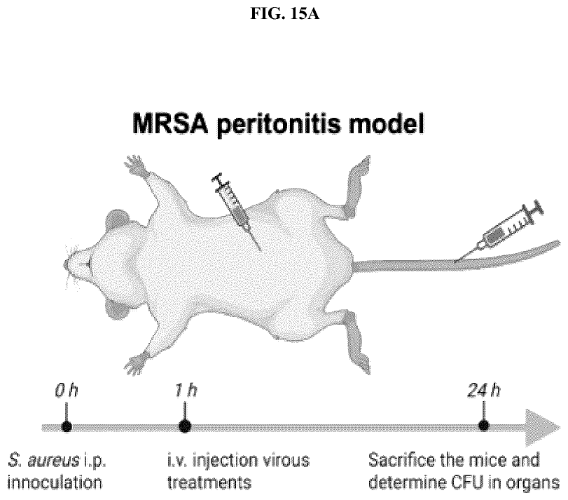

The in vivo antimicrobial efficacy of Hb-Naf@RBCM NP was further evaluated in mice models of MRSA pneumonia, MRSA peritonitis, and MDRSA bacteremia. For the MRSA pneumonia model, MRSA (5×10⁸ colony-forming unit, CFU) was administered intranasally to induce bilateral pneumonia in mice mimicking hospital-acquired pneumonia (FIG. 14A). We first confirmed the establishment of the pneumonia model one hour after inoculation by histopathological studies (FIG. 14B). The infected mice were i.v. injected with free Hb, free naftifine, Hb-Naf NP, and Hb-Naf@RBCM NP (containing 125 mg kg⁻¹ Hb, 25 mg kg⁻¹ naftifine). Hb-Naf@RBCM NP outperformed other treatments, reducing MRSA CFU by over three orders of magnitude (FIG. 14C).

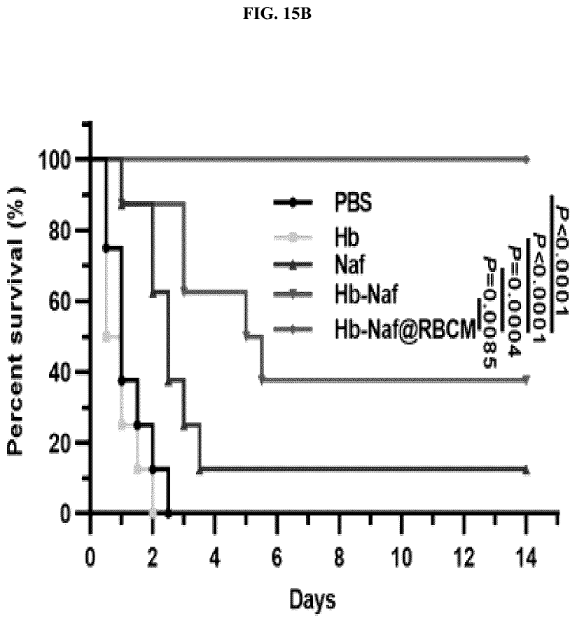

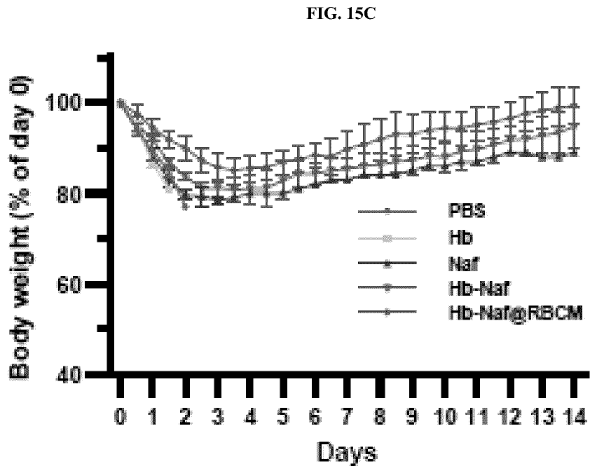

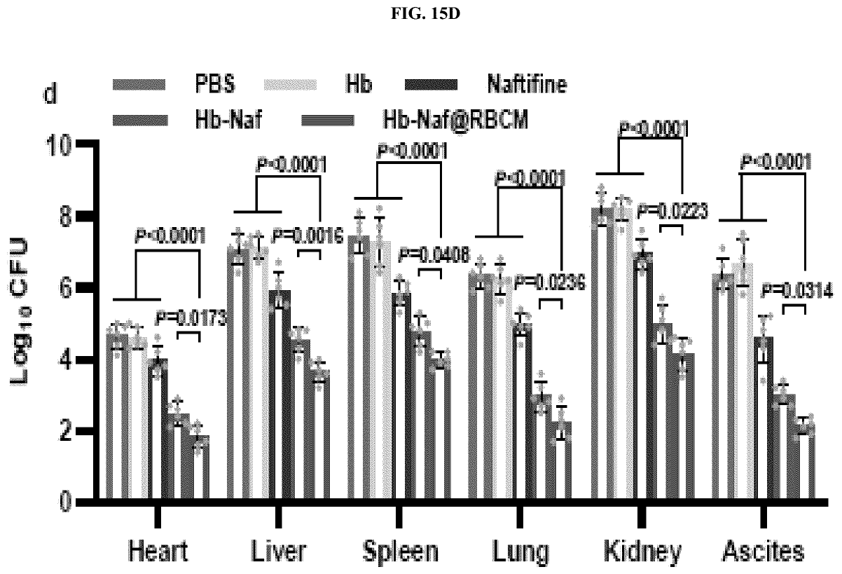

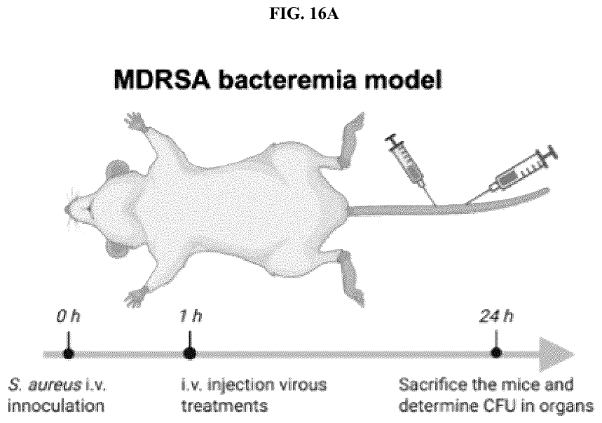

In the MRSA peritonitis model, treatments were i.v. administrated to the infected mice 6 h after MRSA intraperitoneal inoculation (1×10⁸ CFU). Mice survival was monitored for two weeks (FIG. 15A, 15B). Hb-Naf@RBCM NP led to 100% survival, significantly outperforming free naftifine and Hb-Naf NP, which only achieved 12.5% and 37.5% survival rates, respectively. Although body weight loss was initially observed due to infections, mice receiving Hb-Naf@RBCM NP began regaining weight from Day 3 (FIG. 15C). The antimicrobial efficacy was further evaluated by measuring CFUs in organs 12 h post-infection. Hb-Naf@RBCM NP significantly improved therapeutic efficacy compared to other treatments, with Hb-Naf@RBCM NP achieving over 99.9% pathogen elimination in all tested organs (FIG. 15D).

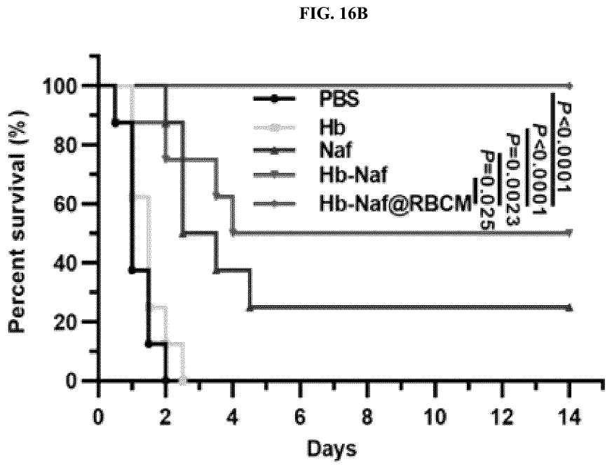

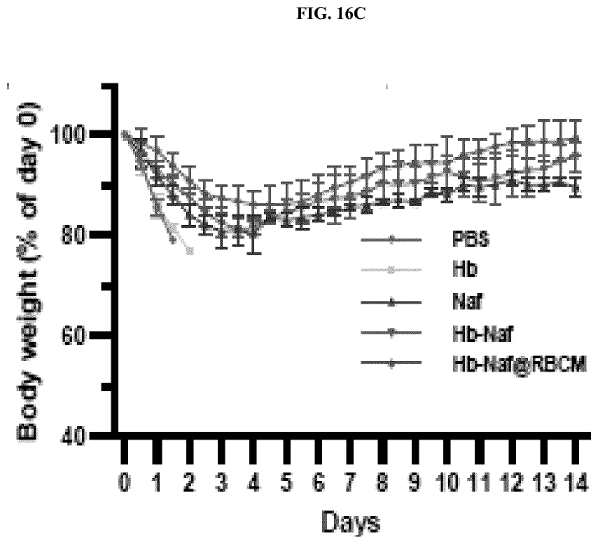

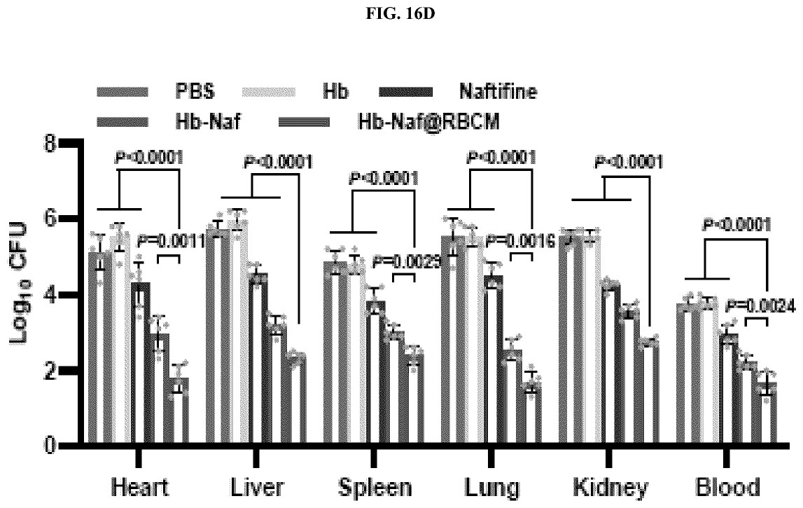

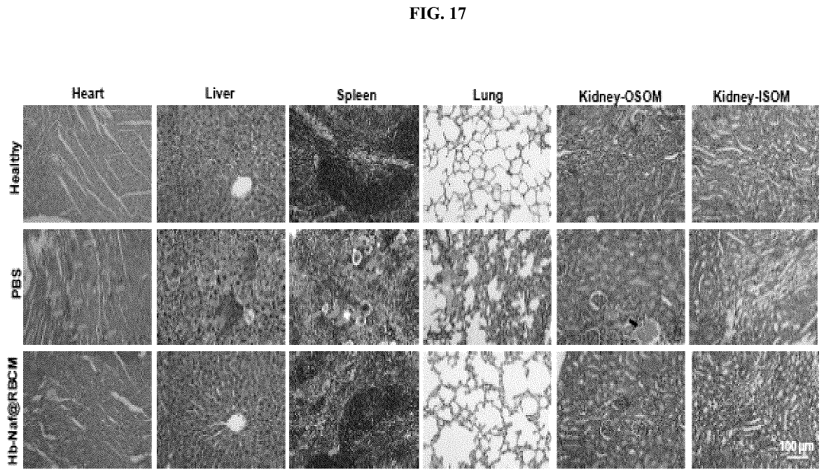

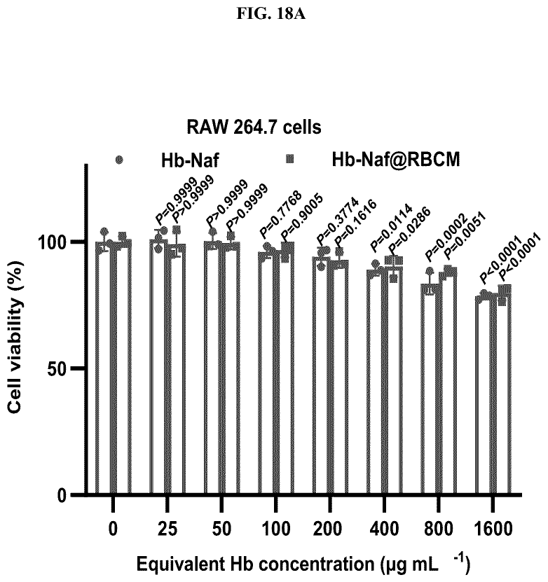

The therapeutic efficacy of Hb-Naf@RBCM NP was also evaluated in the MDRSA bacteremia model, resulting in a 100% 14-day survival rate (FIGS. 16A-16C). Hb-Naf@RBCM NP also significantly lowered bacterial loads in major organs and blood, superior to all other treatments (FIG. 16D). Histopathological analysis showed multiple organ injuries including congestion and dramatical inflammatory cell infiltration in the heart, spotty hepatocellular necrosis, nuclear debris in the spleen, alveolar wall thickening in the lungs, severe congestion in the outer stripe of the outer medulla (OSOM) of the kidney, and tubular necrosis, and cell sloughing in the inner stripe of the outer medulla (ISOM) of the kidney in the PBS-injected mice (FIG. 17). In contrast, Hb-Naf@RBCM NP effectively mitigated these organ injuries. The outstanding therapeutic efficacy exhibited by Hb-Naf@RBCM NP can be attributed to several factors: (1) It had higher accumulation in the infected tissue; (2) It remodeled the bacteria lipid composition to make S. aureus more sensitive to host oxidant killing; and (3) It effectively promoted neutrophil chemotaxis through Hb-Naf@RBCM-induced lipid peroxidation in S. aureus.

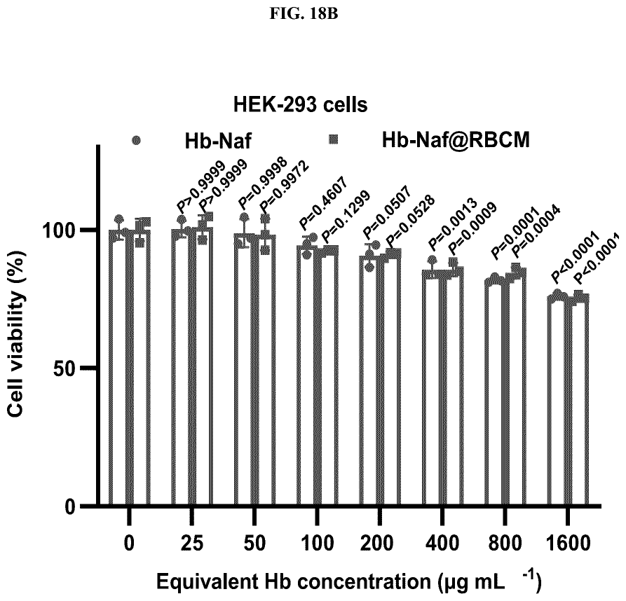

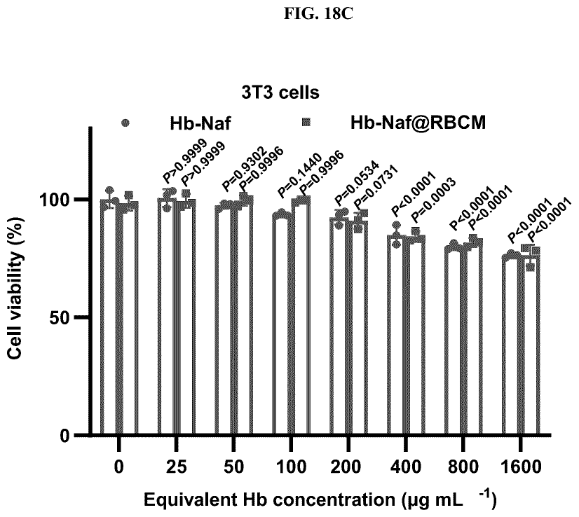

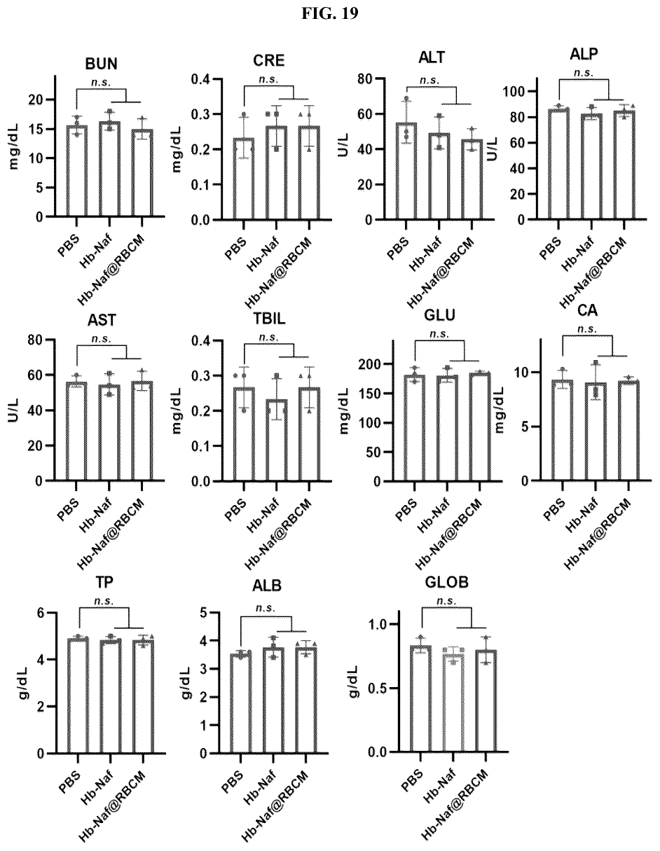

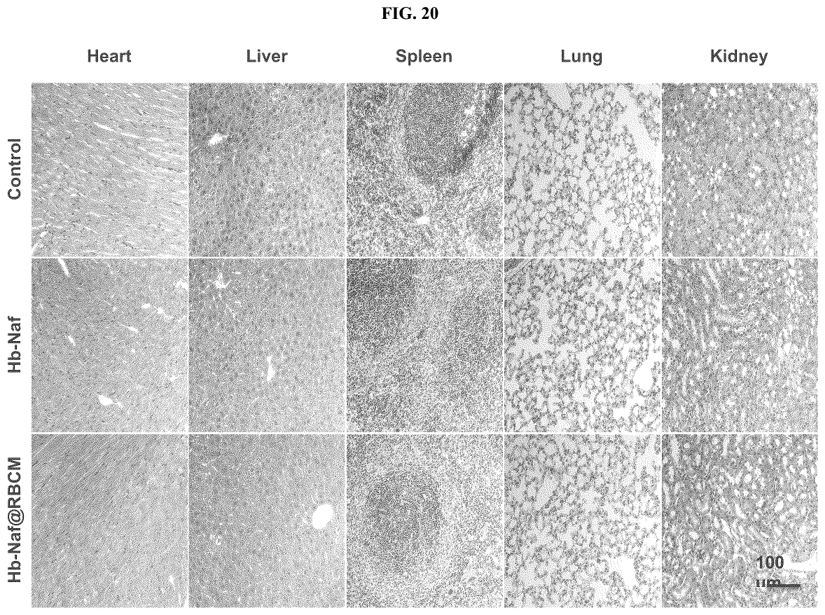

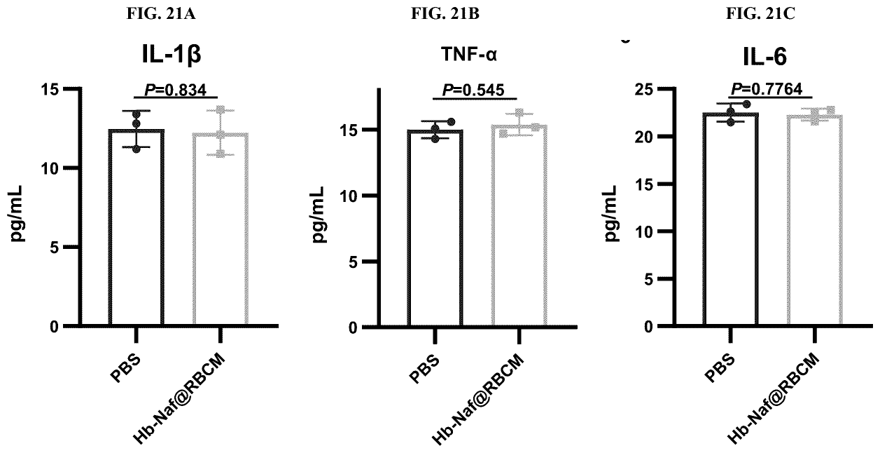

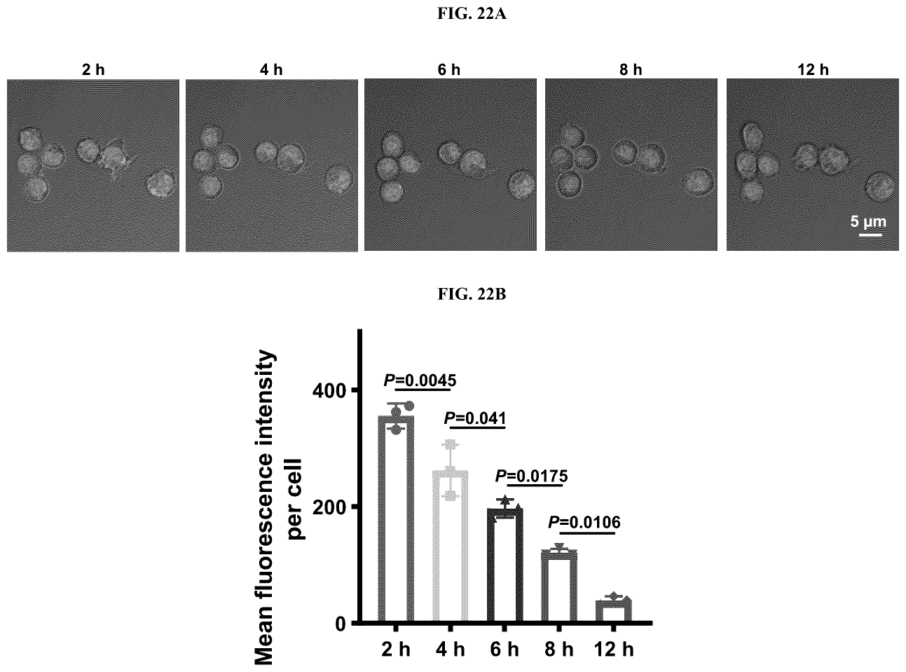

The biocompatibility of Hb-Naf@RBCM NP was also evaluated. In vitro, Hb-Naf@RBCM NP exhibited excellent biocompatibility at the concentration (Hb 200 μg mL⁻¹) used for in vitro studies in murine macrophages (Raw 264.7), human embryonic kidney (HEK 293) cells, and murine fibroblasts (NIH 3T3) (FIGS. 18A-18C). In vivo, the safety of Hb-Naf@RBCM NP was confirmed by three consecutive i.v. administrations performed every other day at the dose used for therapeutic studies. It is worth noting that only a single dose was used for all therapeutic studies. Blood biochemical parameters, histological analysis, and serum proinflammatory cytokine levels of the Hb-Naf@RBCM NP-treated mice did not show any significant differences from those of the PBS control mice (FIGS. 19, 20 and 21A-21C). Additionally, Cy5-labeled Hb-Naf@RBCM NP in Raw 264.7 cells degraded within 12 h post-treatment (FIGS. 22A, 22B). Taken together, these findings demonstrated the safety of Hb-Naf@RBCM NP, which supports its potential clinical translation for treating S. aureus infections.

### EQUIVALENTS

While certain embodiments have been illustrated and described a person with ordinary skill in the art, after reading the foregoing specification, can effect changes, substitutions of equivalents and other types of alterations to the nanoparticles of the present technology or derivatives, prodrugs, or pharmaceutical compositions thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments illustratively described herein may suitably be practiced in the absence of any element or elements, limitation or imitations not specifically disclosed herein, Thus, for example, the terms “comprising,” “including,” “containing,” etc. shall be read expansively and without limitation. Additionally, the phrase “consisting essentially of” will be understood to include those elements specifically recited and those additional elements that do not materially affect the basic and novel characteristics of the claimed technology. The phrase “consisting of” excludes any element not specified. The terms and expressions employed herein have been used as terms of description and not of limitation, and there is no intention in the use or such terms and expressions of excluding any equivalents of the features shown and described or portions thereof, but it is recognized that various modifications are possible within the scope of the claimed technology, Moe specifically, it will be understood that each use of terms such as “comprising,” “consisting essentially of,” or “consisting of”, discloses and provides written description and support for the use any of the other terms with the same or any other embodiment described herein.

In addition, where features or aspects of the disclosure are described in terms of Markush groups, those skilled in the art will recognize that the disclosure is also thereby described in terms of any individual member or subgroup of members of the Markush group, Each of the narrower species and subgeneric groupings failing within the generic disclosure also form part of the technology. This includes the generic description of the technology with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also en compass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety, Definitions that are contained in text incorporated by reference are excluded to the extent that they contradict definitions in this disclosure.

The present technology may include, but is not limited to, the features and combinations of features recited in the following lettered paragraphs, it being understood that the following paragraphs should not be interpreted as limiting the scope of the claims as appended hereto or mandating that all such features must necessarily be included in such claims:
A. A nanoparticle comprising a core and a shell, wherein the core comprises, consists essentially of, or consists of an antimicrobial and hemoglobin, the shell comprises, consists essentially of, or consists of a polyunsaturated fatty acids (PUFA)-containing cell membrane, and the antimicrobial sensitizes bacteria to oxidant killing.
B. The nanoparticle of Paragraph A, wherein the antimicrobial is a redox enzyme inhibitor or an inhibitor of staphyloxanthin biosynthesis.
C. The nanoparticle of Paragraph A or Paragraph B, wherein the antimicrobial is an inhibitor of staphyloxanthin biosynthesis.
D. The nanoparticle of any one of Paragraphs A-C, wherein the antimicrobial is selected from the group consisting of naftifine and ALS 4.
E. The nanoparticle of Paragraph A or Paragraph B, wherein the antimicrobial is a bacterial redox enzyme inhibitor.
F. The nanoparticle of Paragraph E, wherein the antimicrobial is an inhibitor of thioredoxin reductase.
G. The nanoparticle of Paragraph E or Paragraph F, wherein the antimicrobial is auranofin.
H. The nanoparticle of any one of Paragraphs A-G, wherein the antimicrobial comprises about 2.5 wt % to about 60 wt % of the core.
I. The nanoparticle of any one of Paragraphs A-H, further comprising O₂.
J. The nanoparticle of any one of Paragraphs A-I, wherein the PUFA-containing cell membrane is selected from the group consisting of red blood cell membrane (RBCM), macrophage cell membrane, neutrophil cell membrane, mesenchymal stem cell membrane, and platelet cell membrane.
K. The nanoparticle of any one of Paragraphs A-D and H-J, wherein the core comprises, consists essentially of, or consists of naftifine and hemoglobin, wherein the core is coated with RBCM.
L. The nanoparticle of any one of Paragraphs A-K, comprising about 2.5 wt % to about 40 wt % naftifine based on the total weight of the core.
M. The nanoparticle of any one of Paragraphs A-L, wherein the hemoglobin comprises about 60 wt % to about 97.5 wt % based on the total weight of the core.
N. The nanoparticle of any one of Paragraphs A-M, wherein the core includes a molar ratio of hemoglobin to naftifine of about 1:3 to about 1:300.
O. The nanoparticle of Paragraph N, wherein the molar ratio of hemoglobin to naftifine is about 1:10 to about 1:90.
P. The nanoparticle of any one of Paragraphs A-O, having a hydrodynamic diameter of about 20 nm to about 400 nm.
Q. The nanoparticle of any one of Paragraphs A-P, having a hydrodynamic diameter of about 40 nm to about 150 nm.
R. The nanoparticle of any one of Paragraphs A-Q, having a zeta potential of 0 mV to about −40 mV.
S. The nanoparticle of any one of Paragraphs A-R, wherein RBCM comprises about 30 wt % to about 60 wt % of the nanoparticle (based on the total weight of the nanoparticle).
T. A pharmaceutical composition comprising
   a nanoparticle of any one of Paragraphs A-S; and
   a pharmaceutically acceptable carrier or excipient.
U. A method of treatment comprising administering an effective amount of the nanoparticle of any one of Paragraphs A-S to a subject suffering from a bacterial infection and/or administering an effective amount of the pharmaceutical composition of Paragraph T to a subject suffering from a bacterial infection.
V. The method of Paragraph U, wherein the bacterial infection is caused by bacteria selected from the group consisting of S. aureus, E. coli, P. aeruginosa, C. difficile, E. faecium, and K. pneumonia.
W. The method Paragraph V, wherein the bacteria are anti-biotic resistant bacteria.
X. The method of Paragraph V or Paragraph W, wherein the bacteria comprise S. aureus Newman, methicillin-resistant S. aureus (MRSA), and/or multidrug-resistant (MDR) S. aureus.
Y. The method of any one of Paragraphs U-X, wherein the effective amount of the nanoparticle ranges from about 0.1 mg/kg/day to about 500 mg/kg/day.
Z. The method of any one of Paragraphs U-Y, wherein the nanoparticle effects incorporation of PUFA into a membrane of the bacteria, oxidation of hydrogen sulfide produced by the bacteria, and/or potentiates neutrophil-killing of the bacteria causing the bacterial infection.
AA. The method of any one of Paragraphs U-Z, wherein the bacterial infection comprises a bacterial biofilm and/or bacterial persisters.
AB. The method of any one of Paragraphs U-AA, wherein the bacterial infection is a skin infection, bacteremia, bone infection, gastroenteritis, sinus infection, ear infection, urinary tract infection, endocarditis, or pneumonia.
AC. The method of any one of Paragraphs U-AB, further comprising administering an effective amount of an antibiotic to the subject before, during, or after administration of the nanoparticle.
AD. The method of Paragraph AC, wherein the antibiotic is trimethoprim, rifabutin, and/or sulfamethoxazole.

Other embodiments are set forth in the following claims, along with the fill scope of equivalents to which such claims are entitled.

What is claimed is:

1. A nanoparticle comprising a core and a shell, wherein the core comprises an antimicrobial and hemoglobin, the shell comprises a polyunsaturated fatty acids (PUFA)-containing cell membrane, and the antimicrobial sensitizes bacteria to oxidant killing.

2. The nanoparticle of claim 1, wherein the antimicrobial is an inhibitor of staphyloxanthin biosynthesis.

3. The nanoparticle of claim 1, wherein the antimicrobial is selected from the group consisting of naftifine and ALS 4.

4. The nanoparticle of claim 1, wherein the antimicrobial is a bacterial redox enzyme inhibitor.

5. The nanoparticle of claim 4, wherein the antimicrobial is an inhibitor of thioredoxin reductase.

6. The nanoparticle of claim 4, wherein the antimicrobial is auranofin.

7. The nanoparticle of claim 1, wherein the antimicrobial comprises about 2.5 wt % to about 60 wt % of the core.

8. The nanoparticle of claim 1, further comprising O₂.

9. The nanoparticle of claim 1, wherein the PUFA-containing cell membrane is selected from the group consisting of red blood cell membrane (RBCM), macrophage cell membrane, neutrophil cell membrane, mesenchymal stem cell membrane, and platelet cell membrane.

10. The nanoparticle of claim 9, wherein
the core comprises naftifine and hemoglobin; and
the core is coated with RBCM.

11. The nanoparticle of claim 1, comprising about 2.5 wt % to about 40 wt % naftifine based on the total weight of the core.

12. The nanoparticle of claim 1, wherein the hemoglobin comprises about 60 wt % to about 97.5 wt % of the core.

13. The nanoparticle of claim 1, wherein the core includes a molar ratio of hemoglobin to naftifine of about 1:3 to about 1:300.

14. The nanoparticle of claim 1, having a hydrodynamic diameter of about 20 nm to about 400 nm.

15. The nanoparticle of claim 1, having a zeta potential of 0 mV to about −40 mV.

16. The nanoparticle of claim 9, wherein the RBCM comprises about 30 wt % to about 60 wt % of the nanoparticle.

17. A pharmaceutical composition comprising
a nanoparticle of claim 1; and
a pharmaceutically acceptable carrier or excipient.

18. A method of treatment comprising administering an effective amount of the nanoparticle of claim 1 to a subject suffering from a bacterial infection.

19. The method of claim 18, wherein the bacterial infection is caused by bacteria selected from the group consisting of S. aureus, E. coli, P. aeruginosa, C. difficile, E. faecium, and K. pneumonia.

20. The method claim 19, wherein the bacteria are anti-biotic resistant bacteria.

21. The method of claim 18, further comprising administering an effective amount of an antibiotic to the subject before, during, or after administration of the nanoparticle.

22. The method of claim 21, wherein the antibiotic is trimethoprim, rifabutin, and/or sulfamethoxazole.
